(19) United States
(12) Patent Application Publication
(10) Pub. No.: US 2025/0186512 A1
(43) Pub. Date: Jun. 12, 2025

(54) LIVE MYCOPLASMA SYNOVIAE VACCINE

(71) Applicant: UNIVERSITY OF GEORGIA RESEARCH FOUNDATION, INC., Athens, GA (US)

(72) Inventor: Naola M. Ferguson-Noel, Loganville, GA (US)

(21) Appl. No.: 18/846,326

(22) PCT Filed: Mar. 13, 2023
(86) PCT No.: PCT/US2023/015103

§ 371 Date: Sep. 12, 2024

Related U.S. Application Data
(60) Provisional application No. 63/319,532, filed on Mar. 14, 2022.

(51) Int. Cl. A61K 35/74; A61K 39/00; A61K 39/02; A61P 31/04

(52) U.S. Cl. CPC A61K 35/74; A61K 39/0241; A61P 31/04; A61K 2039/52; A61K 2039/552; A61K 2039/575

(57) ABSTRACT

The present invention provides Mycoplasma synoviae strain K5885 as deposited at the ATCC under Patent Designation PTA-127167, and progeny and derivatives thereof, for use as a vaccine for the prevention of virulent Mycoplasma synoviae infections in the birds of the order Galliformes. Also provided are compositions and methods for administration to birds of the order Galliformes.

### CONTINUING APPLICATION DATA

This application claims the benefit of U.S. Provisional Application Ser. No. 63/319,532, filed Mar. 14, 2022, which is incorporated by reference herein.

### BACKGROUND

Mycoplasma synoviae (MS) infection most frequently occurs as a subclinical upper respiratory infection but MS may cause air sac lesions and become systemic and result in infectious synovitis, an acute to chronic infectious disease of chickens and turkeys, involving primarily the synovial membranes of joints and tendon sheaths producing an exudative synovitis, tenovaginitis, or bursitis (Ferguson-Noel and Noormohammadi, “Mycoplasma synoviae infection.” In: Diseases of Poultry. D. E. Swayne, J. R. Glisson, L. R. McDougald, L. K. Nolan, D. L. Suarez and V. Nair, eds. Wiley-Blackwell, Ames, Iowa. pp 900-906. 2013).

M. synoviae is egg transmitted, and the most effective method of control is to select chickens or turkeys from MS-free flocks and the use of effective biosecurity to prevent introduction of the infection (Kleven, 2008, Avian Diseases; 52:367-374). Alternatively, antibiotic treatment and vaccination may be used to ameliorate the effects of MS infection. Although an inactivated, oil emulsion bacterin is commercially available, its role in the control of MS has not been adequately studied. And, while a live temperature-sensitive MS vaccine strain, MS-H, selected by mutagenesis of a field isolate from Australia, is used in many major poultry producing countries, registration in the United States is pending. See Morrow et al., 1998, Avian Diseases; 42:667-670; Markham et al., 1998, Avian Diseases; 42:671-676; Markham et al., 1998, Avian Diseases; 42:677-681; and Markham et al., 1998, Avian Diseases; 42:682-689. Thus, there is a need for additional vaccines for the control of Mycoplasma synoviae infection in poultry.

### SUMMARY OF THE INVENTION

The present invention includes an isolated Mycoplasma synoviae strain, wherein the isolated Mycoplasma synoviae strain is the K5885 Mycoplasma synoviae strain deposited at the ATCC under Patent Designation PTA-127167, or a progeny or derivative thereof. In some aspects, the present invention includes a composition including the isolated Mycoplasma synoviae strain deposited at the ATCC under Patent Designation PTA-127167, or a progeny or derivative thereof. In some aspects, the composition may include water. In some aspects, the composition may include a pharmaceutically acceptable carrier. In some aspects, the composition may include an adjuvant. In some aspects, the composition may be formulated for intranasal, intraocular, oral, mucosal, intramuscular, subcutaneous, or in ovo administration. In some aspects, the composition may be formulated for spraying or aerosolizing.

The present invention includes an essentially biologically pure culture of the K5885 Mycoplasma synoviae strain deposited at the ATCC under Patent Designation PTA-127167, or a progeny or derivative thereof. In some aspects, the present invention includes a composition including the essentially biologically pure culture of the K5885 Mycoplasma synoviae strain deposited at the ATCC under Patent Designation PTA-127167, or a progeny or derivative thereof. In some aspects, the composition may include water. In some aspects, the composition may include a pharmaceutically acceptable carrier. In some aspects, the composition may include an adjuvant. In some aspects, the composition may be formulated for intranasal, intraocular, oral, mucosal, intramuscular, subcutaneous, or in ovo administration. In some aspects, the composition may be formulated for spraying or aerosolizing.

The present invention includes a vaccine including an isolated Mycoplasma synoviae strain as described herein, an essentially biologically pure culture of the K5885 Mycoplasma synoviae strain as described herein, or a composition as described herein. In some aspects, the vaccine reduces one or more of the clinical signs induced by Mycoplasma synoviae infection in poultry. In some aspects, the vaccine reduces the susceptibility of a birds of the order Galliformes to disease induced by Mycoplasma synoviae.

The present invention includes a live vaccine for birds of the order Galliformes, the vaccine including an amount of the K5885 Mycoplasma synoviae strain deposited at the ATCC under Patent Deposit Designation PTA-127167 or a progeny or derivative thereof, sufficient to protect the birds from disease induced by Mycoplasma synoviae, and a pharmaceutically acceptable carrier.

In some aspects, an isolated Mycoplasma synoviae strain as described herein, a composition as described herein, or a vaccine as described herein may be lyophilized, freeze dried, frozen, or an effervescent tablet.

The present invention includes a kit including an isolated Mycoplasma synoviae strain as described herein, a composition as described herein, or a vaccine as described herein and printed instructions, wherein the contents of the kit are contained within packaging material.

The present invention includes an effervescent tablet including an isolated Mycoplasma synoviae stain as described herein, a composition as described herein, or a vaccine as described herein.

The present invention includes a method of producing an immune response to Mycoplasma synoviae in a bird, the method including administering an isolated Mycoplasma synoviae stain as described herein, a composition as described herein, or a vaccine as described herein to the bird. In some aspect, with a method described herein, administration is intranasal, intraocular, oral, mucosal, intramuscular, subcutaneous, or in ovo. In some aspect, with a method described herein, administration is by eye drop, by aerosol, or by drinking water. In some aspect, with a method described herein the bird is of the order Galliformes. In some aspect, with a method described herein, the bird is a chicken or a turkey.

The present invention includes a method for reducing susceptibility of a bird against disease induced by Mycoplasma synoviae, the method including administering an isolated Mycoplasma synoviae stain as described herein, a composition as described herein, or a vaccine as described herein to the bird. In some aspect, with a method described herein, administration is intranasal, intraocular, oral, mucosal, intramuscular, subcutaneous, or in ovo. In some aspect, with a method described herein, administration is by eye drop, by aerosol, or by drinking water. In some aspect, with a method described herein the bird is of the order Galliformes. In some aspect, with a method described herein, the bird is a chicken or a turkey.

The present invention includes a method for protecting a bird against Mycoplasma synoviae infection, the method including administering an isolated Mycoplasma synoviae stain as described herein, a composition as described herein, or a vaccine as described herein the bird. In some aspect, with a method described herein, administration is intranasal, intraocular, oral, mucosal, intramuscular, subcutaneous, or in ovo. In some aspect, with a method described herein, administration is by eye drop, by aerosol, or by drinking water. In some aspect, with a method described herein the bird is of the order Galliformes. In some aspect, with a method described herein, the bird is a chicken or a turkey.

The present invention includes a method of reducing one or more clinical signs induced by a Mycoplasma synoviae infection in a bird, the method including administering an effective amount of an isolated Mycoplasma synoviae stain as described herein, a composition as described herein, or a vaccine as described herein to the bird. In some aspect, with a method described herein, a clinical sign comprises body weight suppression, decrease in egg production, mortality, upper respiratory infection, lameness, swelling of the joints, ovarian regression, air sac lesions, tracheal lesions, footpad lesions, and/or eggshell apex abnormalities. In some aspect, with a method described herein, administration is intranasal, intraocular, oral, mucosal, intramuscular, subcutaneous, or in ovo. In some aspect, with a method described herein, administration is by eye drop, by aerosol, or by drinking water. In some aspect, with a method described herein the bird is of the order Galliformes. In some aspect, with a method described herein, the bird is a chicken or a turkey.

As used herein, “isolated” refers to material removed from its original environment (e.g., the natural environment if it is naturally occurring), and thus is altered “by the hand of man” from its natural state.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

In several places throughout the application, guidance is provided through lists of examples, which examples can be used in various combinations. In each instance, the recited list serves only as a representative group and should not be interpreted as an exclusive list. It is to be understood that the particular examples, materials, amounts, and procedures are to be interpreted broadly in accordance with the scope and spirit of the invention as set forth herein.

All headings throughout are for the convenience of the reader and should not be used to limit the meaning of the text that follows the heading, unless so specified.

### BRIEF DESCRIPTION OF THE FIGURES

FIG. 1. Results of Trial 1 (Safety I). Mean tracheal mucosa measurements of chickens at 10 and 14 DPI with K5885A, K4971B, K5805A or K1968. Different lowercase superscripts are significantly different (P<0.05) at a specific time point (10 or 14 DPI).

FIG. 2. Results of Trial 1 (Safety I). Air sac lesions of from chickens at 10 and 14 DPI with K5885A, K4971B, K5805A or K1968. Different lowercase superscripts are significantly different (P<0.05) at a specific time point (10 or 14 DPI).

FIG. 3. Results of Trial 3 (Safety II). Air sac lesion scores from chickens at 14 DPI with K5885A or K6677.

FIG. 4. Results of Trial 3 (Safety II). Foot pad lesion scores from chickens at 14 DPI with K5885A or K6677. Different lowercase superscripts are significantly different (P<0.05).

FIG. 5. Results of Trial 4 (Efficacy II). Air sac lesion scores of vaccinated and non-vaccinated chickens 14 days post challenge with K6677. Different lower case superscripts are significantly different (P<0.05).

FIG. 6. Results of Trial 4 (Efficacy II). Foot pad lesion scores of vaccinated and non-vaccinated chickens 14 days post challenge with K6677.

FIG. 7. Results of Trial 5 (Efficacy III). Air sac lesion scores of vaccinated and nonvaccinated chickens 14 days post challenge with K6677A. Different lowercase superscripts are significantly different (P<0.05).

FIG. 8. Results of Trial 5 (Efficacy III). Foot pad lesion scores of vaccinated and nonvaccinated chickens 14 days post challenge with K6677. Different lower case superscripts are significantly different (P<0.05).

FIG. 9. Air sac lesion scores among the different groups from 2 to 6 weeks post-vaccination. * Means differences are significant at P<0.05. NV/NC: non-vaccinated non-challenged. NV/C-K: non-vaccinated K6677-challenged. NV/C-W: non-vaccinated WVU 1853-challenged. V/C-K: vaccinated K6677-challenged. V/C-W: vaccinated WVU 1853-challenged. V/NC: vaccinated non-challenged.

FIG. 10. Footpad lesion scores among the different groups from 2 to 6 weeks post-vaccination. * means differences are significant at P<0.05. ** means differences are significant at P<0.005. NV/NC: non-vaccinated non-challenged. NV/C-K: non-vaccinated K6677-challenged. NV/C-W: non-vaccinated WVU 1853-challenged. V/C-K: vaccinated K6677-challenged. V/C-W: vaccinated WVU 1853-challenged. V/NC: vaccinated non-challenged.

FIG. 11. Percentages of ovarian regression among the different groups from 2-6 weeks post-vaccination. * Means differences are significant at P<0.05. NV/NC: non-vaccinated non-challenged. NV/C-K: non-vaccinated K6677-challenged. NV/C-W: non-vaccinated WVU 1853-challenged. V/C-K: vaccinated K6677-challenged. V/C-W: vaccinated WVU 1853-challenged. V/NC: vaccinated non-challenged.

FIG. 12. Mean genome copy number of MS in the trachea of different groups from 2 to 6 weeks post-vaccination. NV/NC: non-vaccinated non-challenged. NV/C-K: non-vaccinated K6677-challenged. NV/C-W: non-vaccinated WVU 1853-challenged. V/C-K: vaccinated K6677-challenged. V/C-W: vaccinated WVU 1853-challenged. V/NC: vaccinated non-challenged.

FIG. 13. Mean genome copy number of K5885 and K6677 in different groups from 2 to 6 weeks post-vaccination. * means differences are significant at P<0.05. ** means differences are significant at P<0.005. *** means differences are significant at P<0.0005. NV/NC: non-vaccinated non-challenged. NV/C-K: non-vaccinated K6677-challenged. NV/C-W: non-vaccinated WVU 1853-challenged. V/C-K: vaccinated K6677-challenged. V/C-W: vaccinated WVU 1853-challenged. V/NC: vaccinated non-challenged.

FIG. 14. Percentage egg production of the chickens in all groups from the onset of egg production to 6 WPC. NV/NC: non-vaccinated non-challenged. NV/C-K: non-vaccinated K6677-challenged. NV/C-W: non-vaccinated WVU 1853-challenged. V/C-K: vaccinated K6677-challenged. V/C-W: vaccinated WVU 1853-challenged. V/NC: vaccinated non-challenged.

### DETAILED DESCRIPTION OF ILLUSTRATIVE EMBODIMENTS

The bacteria Mycoplasma synoviae is a member of the mycoplasma genus. It causes disease in the joints, bones, and respiratory system of birds. It is found throughout the world and infection may be referred to as infectious synovitis, avian mycoplasmosis, infectious sinusitis, or mycoplasma arthritis. It is of economic importance because infection can cause a drop in egg production. The disease is seen primarily in chickens and turkeys, but ducks, geese, guinea fowl, parrots, pheasants, and quail may also be susceptible. Transmission occurs both vertically and horizontally. Mycoplasma synoviae most commonly causes subclinical upper respiratory infections in chickens, turkeys, and other avian species, though it can also cause exudative tendinitis and synovitis, known as infectious synovitis.

The present invention provides Mycoplasma synoviae (MS) strain K5885 and progeny and derivatives thereof that are immunogenic and stable when administered as live formulations. Formulations of the Mycoplasma synoviae of the present invention are safe and efficacious to inhibit Mycoplasma synoviae infections and will be useful in reducing the incidence and severity of disease of Mycoplasma synoviae infections in birds.

Mycoplasma synoviae strain K5885 was deposited with the American Type Culture Collection (ATCC®), 10801 University Boulevard, Manassas, VA 20110-2209, USA, as PTA-127167 on Nov. 24, 2021. This strain was deposited in accordance with the Budapest Treaty on the International Recognition of the Deposit of Microorganisms for the Purposes of Patent Procedure. Mycoplasma synoviae strain K5885 as deposited with the ATCC© as PTA-127167 is also referred to herein as Mycoplasma synoviae strain K5885, Mycoplasma synoviae strain K5885A, MS strain K5885, MS strain K5885A, K5885, K5885A, MS strain K5885 ATCC PTA-127167, MS strain K5885A ATCC PTA-127167, K5885 ATCC PTA-127167, K5885A ATCC PTA-127167, MS strain K5885 PTA-127167, MS strain K5885A PTA-127167, K5885 PTA-127167, K5885A PTA-127167, ATCC PTA-127167, and PTA-127167.

The present invention includes isolated Mycoplasma synoviae (MS) strain K5885 with the ATCC Patent Deposit Designation PTA-127167. As used herein, “isolated” refers to material removed from its original environment (e.g., the natural environment if it is naturally occurring), and thus is altered “by the hand of man” from its natural state. Also included are biologically pure cultures of Mycoplasma synoviae (MS) strain K5885 with the ATCC Patent Deposit Designation PTA-127167.

Also included in the present invention are isolated progeny and isolated derivatives of Mycoplasma synoviae strain K5885 deposited with the ATCC Patent Deposit Designation as PTA-127167 with equivalent or similar biological, serological, and/or genetic characteristics. As used herein, serological, biological, and genetic characteristics may include one or more of the characteristics described in the data in the Examples and Figures included herewith. More particularly, progeny or derivative of the K5885 strain deposited with the ATCC as PTA-127167 may retain the particularly favorable protective properties belonging to the present invention. Progeny or derivatives of Mycoplasma synoviae strain K5885 deposited with the ATCC as PTA-127167 may be obtained by any of the various methods for propagating Mycoplasma synoviae known in the art, including, but not limited to, for example, in vitro culture or back passage in a bird. Derivatives of Mycoplasma synoviae strain K5885 ATCC PTA-127167 may include genetically modified versions of the deposited MS K5885 strain. Such manipulations include, but are not limited to, mutagenizing the MS strain, or introducing genes or gene cassettes encoding alternative proteins or nonfunctional proteins, or noncoding nucleotide sequences into the MS organism.

A Mycoplasma synoviae (MS) strain K5885 isolate as described herein may be propagated by conventional methods, including, but not limited to, any of those described in the examples section included herewith. For example, a Mycoplasma synoviae strain of the present invention may be cultured as described in more detail in Avian Mycoplasmas, Harry W. Yoder Jr., in Diagnostic Procedure in Veterinary Bacteriology and Mycology (Fifth Edition), 1990. Briefly, Mycoplasma synoviae may be cultured at 37° C. in Frey's broth or agar supplemented with 10-15% normal swine serum. The swine serum may be heat inactivated at 56° C. for 30 min. In addition, M. synoviae may require the addition of 0.1% reduced nicotinamide adenine dinucleotide (NAD) to broth and agar media.

MS strain K5885 and progeny and derivatives thereof may be identified and differentiated from other M. synoviae strains using any of the many techniques that have been developed for the differentiation of M. synoviae strains, including, for example, direct immunofluorescence and real-time quantitative PCR (qPCR) (Raviv and Kleven, 2009, Avian Dis; 53:103-107), multi-locus sequence typing (MLST) (Dijkman et al., 2016, Avian Pathol; 45(4):426-442 and El-Gazzar et al., 2017, Avian Dis: 61(1):25-32), and PCR analysis of the lipoprotein and hemagglutinin A (vlhA) gene (Wetzel et al., 2010, Avian Dis 54(4): 1292-1297).

The present invention includes compositions and vaccines of the M. synoviae isolates and progeny and derivatives thereof as described herein. In preferred embodiments, the M. synoviae isolate is live. In some embodiments, the M. synoviae isolate may be inactivated or killed. A M. synoviae strain and compositions and vaccines thereof of the present invention may be stored until use in any of a variety of forms. For example, such materials, may be lyophilized or freeze dried and may be rehydrated for use. In some embodiments, a M. synoviae strain or composition or vaccine thereof may be frozen.

In some embodiments, a M. synoviae isolate or composition or vaccine thereof may be formulated as an effervescent table. Such effervescent tablets may, for example, be packaged in lightweight aluminum blisters. The table may be dissolved in water and administered, for example, orally, nasally, or by aerosol spray, whereby droplets enter via the mucus membranes of the birds.

Compositions and vaccines of the present invention may include, for example, water or culture medium. Such compositions and vaccines may include one or more suitable pharmaceutically acceptable carriers, stabilizers, preservatives, diluents, and/or buffers. Suitable stabilizers include, for example, SPGA, carbohydrates (such as sorbitol, mannitol, starch, sucrose, dextrin, or glucose), or proteins (such as albumin or casein). A stabilizer is particularly advantageous when a dry vaccine preparation is prepared by lyophilization. Suitable preservatives include, for example, thimerosal, merthiolate, and gentamicin. Diluents include, but are not limited to, water, aqueous buffer (such as buffered saline), alcohols, and polyols (such as glycerol).

A composition or vaccine of the present invention may also include one or more compounds with adjuvant activity. Suitable compounds or compositions for this purpose include aluminum hydroxide, aluminum phosphate, aluminum oxide, plant oils, animal oils, oil-in-water or water-in-oil emulsion based on, for example a mineral oil, such as Bayol F™ or Marcol 52™ Complete Freund's adjuvant, incomplete Freund's adjuvant, or a vegetable oil such as vitamin E acetate, and saponins.

A composition or vaccine of the present invention may further include one or more immunogens derived from other pathogens infectious to poultry. Such immunogens may be derived from, for example, Mycoplasma gallisepticum (MG), Marek's disease virus (MDV), infectious bronchitis virus (IBV), Newcastle disease virus (NDV), egg drop syndrome (EDS) virus, turkey rhinotracheitis virus (TRTV), poxvirus, reovirus, chicken parvovirus, and avian nephritis virus (including, but not limited to ANV-1 and ANV-2).

Compositions and vaccines of the present invention may be substantially pure. As used herein, “substantially pure” will mean material essentially free of macromolecules or other biological entities that would normally be found with it in nature.

Compositions and vaccines of the present invention may be administered to birds of any of a variety of avian species that are susceptible to Mycoplasma synoviae infection, including, but not limited to, poultry, birds of the order Galliformes, and exotic bird species. Birds of the order Galliformes include, but are not limited to, chickens, turkeys, grouse, quails, and pheasants. As used herein, poultry includes domesticated birds that are kept for the purpose of collecting their eggs or killing for their meat and/or feathers. These most typically are members of the superorder Galloanserae (fowl), especially the order Galliformes (which includes, for example, chickens, quail, turkeys, and grouse) and the family Anatidae (in order Anserifornies), commonly known as “waterfowl” (including, for example, ducks, geese, and swans). Poultry may also include other birds which are killed for their meat, such as pigeons or doves or birds considered to be game, like pheasants. Chickens include, but are not limited to, hens, roosters, broilers, roasters, layers, breeders, the offspring of breeder hens, and layers. As used herein, the term “susceptible to” means the possibility or actuality of a detrimental response to the referenced microorganism, such as, for example, reduced vigor or a failure to thrive, when compared to a non-susceptible individuals or groups, and/or one or more pathological state(s) indicative of Mycoplasma synoviae infection.

Compositions and vaccines of the present invention may be formulated for delivery by any of a variety of routes known in the veterinary arts, including, but not limited to, for example, mucosal, intranasal, intraocular, or oral administration. Compositions and vaccines of the present invention may be formulated for delivery to the respiratory mucosa and may be administered such that it is immediately or eventually brought into contact with the bird's respiratory mucosal membranes. A composition or vaccine of the present invention may be administered by any suitable known method of inoculating poultry including, but not limited to, nasally, ocularly, by injection, in drinking water, in the feed, by exposure, in ovo, maternally, by respiratory inhalation, and the like. When administered by injection, the immunogenic composition or vaccine may be administered parenterally. Parenteral administration includes, for example, administration by intravenous, subcutaneous, intramuscular, or intraperitoneal injection.

A composition or vaccine may be formulated for administered by mass administration techniques such as by placing the vaccine in drinking water or by spraying or aerosolizing. A composition may be administered by spraying an individual or the flock with a solution, such aerosol delivery may involve the administration of the composition incorporated in small liquid particles. Such spray-type particles may have a droplet size ranging from between about 10 to about 100 microns, more preferably, a droplet size from between about <1 to about 50 microns. For the generation of the small particles, conventional spray-apparatus and aerosol generators may be used, such as the commercially available spray generators for knapsack spray, hatchery spray and atomist spray. Administration through drinking water may can be carried out using conventional apparatus.

A composition or vaccine of the present invention may be administered to poultry before or after hatching. In ovo vaccination may take place, for example, at about 13 days, about 14 days, about 15 days, about 16 days, about 17 days, about 18 days, about 19 days, about 20 days, or at any range thereof. For in ovo delivery, laying stock or reproduction stock may be vaccinated, for example, at about 6-12 weeks of age and boosted at about 16-20 weeks of age. Such laying stock or reproduction stock may be vaccinated at about 6, at about 7, at about 8, at about 9, at about 10, at about 11, or at about 12 weeks of age. Also, in some embodiments, such laying stock or reproduction stock may be vaccinated within about the first two weeks of age. Such laying stock or reproduction stock may be boosted at about 16, at about 17, at about 18, at about 19, or at about 20 weeks of age. The offspring of such laying stock or reproduction stock may demonstrate an antibody titer to Mycoplasma synoviae, which may prevent or mitigate the symptoms of a Mycoplasma synoviae infection in the offspring

Poultry may receive a composition or vaccine as described herein at a variety of ages. With delivery after hatching, materials may be delivered at any suitable age, including, but not limited to, about one to three days old, about one week after hatching, about two weeks after hatching, about three weeks after hatching, about four weeks after hatching, about five weeks after hatching, about six weeks after hatching, or any range thereof. The chickens may be vaccinated only once. Or, if two doses of vaccine are used, the first is given, for example, when the chickens are 3 days to a week old and subsequently after a further 1-10 weeks.

Multiple doses of the composition can be administered throughout the life of the chicken. As maternal immunity is a primary source of providing protection to broiler progeny, breeder chickens are typically vaccinated, although broiler chickens can be vaccinated if so desired.

Compositions and vaccines of the present invention may be adjusted to include a designated concentration of Mycoplasma synoviae. Organisms may be measured as color changing units. Color changing units, also referred to herein as “ccu,” of Mycoplasma synoviae can be quantified using established standard methodology, including, for example, protocols set forth in Rodwell and Whitcomb (In “Methods in Mycoplasmology,” Eds. Razin and Tully, 1993). For example, compositions or vaccines may have concentration of about 50, about 100, about 1×10² ccu/ml, about 2.5×10² ccu/ml, about 5×10² ccu/ml, about 1×10³ ccu/ml, about 2.5×10³ ccu/ml, about 5×10³ ccu/ml, about 1×10⁴ ccu/ml, about 2.5×10⁴ ccu/ml, about 5×10⁴ ccu/ml, about 1×10⁵ ccu/ml, about 2.5×10⁵ ccu/ml, about 5×10⁵ ccu/ml, about 1×10⁶ ccu/ml, about 2.5×10⁶ ccu/ml, about 5×10⁶ ccu/ml, about 1×10⁸ ccu/ml, about 2.5×10⁷ ccu/ml, about 5×10⁷ ccu/ml, 1×10⁸ ccu/ml, about 2.5×10⁸ ccu/ml, about 5×10⁸ ccu/ml, about 1×10⁹ ccu/ml, about 2.5×10⁹ ccu/ml, or about 5×10⁹ ccu/ml, and any range thereof (such as, for example, about 1×10⁵ ccu/ml to about 1×10⁶ ccu/ml) may be used. In some applications, an effective amount may be administered to a single bird at one drop per eye per bird. One drop may be approximately 0.05 ml to 0.1 ml.

Mycoplasma synoviae strains of the present invention may be administered to birds to reduce susceptibility to Mycoplasma synoviae infection. With such administration, the materials do not result in significant clinical signs or lesions indicative of Mycoplasma synoviae. Accordingly, it is an object of the present invention to provide immunological materials that with administration do not result in significant clinical signs or lesions indicative of MS disease. It is another object to provide immunological materials of low virulence.

The present invention includes a method of producing an anti-MS immune response in poultry, the method including administering a Mycoplasma synoviae strain, composition, or vaccine as described herein. In some aspects, immunity includes humoral and/or cellular immunity. With a humoral response, anti-MS antibodies may be measured, for example, by the serum plate agglutination (SPA) test (using for example, commercial antigen (Charles River Laboratories International, Inc., Wilmington, MA)); the hemagglutination inhibition (HI) test (using for example, antigen prepared from the WVU1853 strain); and the enzyme-linked immunosorbent assay (ELISA) test (using for example, a commercial kit (IDEXX, Westbrook, Maine)). The SPA and HI test procedures are described in more detail in Ferguson-Noel et al. (Ferguson-Noel, N., and S. H. Kleven Mycoplasma species. In: A Laboratory Manual for the Isolation, Identification and Characterization of Avian Pathogens, Sixth ed. S. M. Williams, L. Dufour-Zavala, M. W. Jackwood, M. D. Lee, B. Lupiani, W. M. Reed, E. Spackman, and P. R. Woolcock, eds. American Association of Avian Pathologists. pp 63-70. 2016). In some aspects, immunity includes mucosal immunity.

Administration of an isolated Mycoplasma synoviae strain, composition, or vaccine as described herein may result in the reduction, inhibition, or prevention of one or more of the disease manifestations of challenge with a further infection with MS, including one or more of the disease manifestations of infectious MS. Such symptoms may include one or more of body weight suppression, decrease in egg production, mortality, clinical signs, such as for example, upper respiratory infection, lameness, swelling of the joints, and/or ovarian regression, and/or histopathological indications, such as, for example, air sac lesions, tracheal lesions, and/or footpad lesions. The present invention includes a method of reducing, inhibiting, or preventing an MS infection in poultry, the method including administering an isolated Mycoplasma synoviae strain, composition or vaccine as described herein.

The invention also provides a kit including Mycoplasma synoviae strain K5885 and/or a progeny or derivative thereof as described herein. The kit may include one or more containers filled with a Mycoplasma synoviae of the present invention. The Mycoplasma synoviae strain K5885 may be lyophilized. The kit may include additional, separate containers of other strains of Mycoplasma synoviae or other pathogens of poultry. Additionally, the kit may include other reagents such as buffers and solutions needed to practice the invention are also included. Optionally associated with such container(s) can be a notice or printed instructions. A kit of the present invention may include “packaging material.” As used herein, the term “packaging material” refers to one or more physical structures used to house the contents of the kit. Packaging material is constructed by well-known methods, preferably to provide a sterile, contaminant-free environment. Packaging material may be a solid matrix or a material such as glass, plastic, paper, foil, and the like. Thus, for example, a package can be a glass or plastic vial used to contain ccu quantities of Mycoplasma synoviae strain K5885.

Exemplary Embodiments of the present invention include, but are not limited to, the following.

1. An isolated Mycoplasma synoviae strain, wherein the isolated Mycoplasma synoviae strain is the K5885 Mycoplasma synoviae strain deposited at the ATCC under Patent Designation PTA-127167, or a progeny or derivative thereof.

2. An essentially biologically pure culture of the K5885 Mycoplasma synoviae strain deposited at the ATCC under Patent Designation PTA-127167.

3. A composition comprising the isolated Mycoplasma synoviae of Embodiment 1 or 2.

4. The composition of Embodiment 3 comprising water.

5. The composition of Embodiment 3 or 4 comprising a pharmaceutically acceptable carrier.

6. The composition of any one of Embodiments 3 to 5 comprising an adjuvant.

7. The composition of any one of Embodiments 3 to 6, wherein the composition is formulated for intranasal, intraocular, oral, mucosal, intramuscular, subcutaneous, or in ovo administration.

8. The composition of any one of Embodiment 3 to 7, wherein the composition is formulated for spraying or aerosolizing.

9. A vaccine comprising the isolated Mycoplasma synoviae of Embodiment 1 or 2 or the composition of any one of Embodiments 3 to 8.

10. The vaccine of Embodiment 9, wherein the vaccine reduces one or more of the clinical signs induced by Mycoplasma synoviae infection in poultry.

11. The vaccine of Embodiment 9 or 10, wherein the vaccine reduces the susceptibility of a birds of the order Galliformes to disease induced by Mycoplasma synoviae.

12. A live vaccine for birds of the order Galliformes, the vaccine comprising an amount of the K5885 Mycoplasma synoviae strain deposited at the ATCC under Patent Deposit Designation PTA-127167 or a progeny or derivative thereof, sufficient to protect the birds from disease induced by Mycoplasma synoviae, and a pharmaceutically acceptable carrier.

13. The isolated Mycoplasma synoviae of Embodiment 1 or 2, the composition of any one of Embodiments 3 to 8, or the vaccine of any one of Embodiments 9 to 12, wherein the isolated Mycoplasma synoviae, composition, or vaccine is lyophilized, freeze dried, frozen, or an effervescent tablet.

14. A kit comprising the isolated Mycoplasma synoviae of Embodiment 1 or 2, the composition of any one of Embodiments 3 to 8, or the vaccine of any one of Embodiments 9 to 12 and printed instructions, wherein the contents of the kit are contained within packaging material.

15. An effervescent tablet comprising the isolated Mycoplasma synoviae of Embodiment 1 or 2, the composition of any one of Embodiments 3 to 8, or the vaccine of any one of Embodiments 9 to 12.

16. A method of producing an immune response to Mycoplasma synoviae in a bird, the method comprising administering the isolated Mycoplasma synoviae of Embodiment 1 or 2, the composition of any one of Embodiments 3 to 8, or the vaccine of any one of Embodiments 9 to 12 to the bird.

17. A method for reducing susceptibility of a bird against disease induced by Mycoplasma synoviae, the method comprising administering the isolated Mycoplasma synoviae of Embodiment 1 or 2, the composition of any one of Embodiments 3 to 8, or the vaccine of any one of Embodiments 9 to 12 to the bird.

18. A method for protecting a bird against Mycoplasma synoviae infection, the method comprising administering the isolated Mycoplasma synoviae of Embodiment 1 or 2, the composition of any one of Embodiments 3 to 8, or the vaccine of any one of Embodiments 9 to 12 to the bird.

19. A method of reducing one or more clinical signs induced by a Mycoplasma synoviae infection in a bird, the method comprising administering an effective amount of the isolated Mycoplasma synoviae of Embodiment 1 or 2, the composition of any one of Embodiments 3 to 8, or the vaccine of any one of Embodiments 9 to 12 to the bird.

20. The method of Embodiments 19, wherein the one or more clinical signs comprises body weight suppression, decrease in egg production, mortality, upper respiratory infection, lameness, swelling of the joints, ovarian regression, air sac lesions, tracheal lesions, footpad lesions, and/or eggshell apex abnormalities.

21. The method of any one of Embodiments 16 to 20, wherein administration is intranasal, intraocular, oral, mucosal, intramuscular, subcutaneous, or in ovo.

22. The method of any one of Embodiments 16 to 20, wherein administration is by eye drop, by aerosol, or by drinking water

23. The method of any one of Embodiments 16 to 22, wherein the bird comprises a bird of the order Galliformes.

24. The method of any one of Embodiments 16 to 23, wherein the bird comprises a chicken or a turkey.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Evaluation of a Potential Live Mycoplasma synoviae Vaccine in Chickens

This example has identified a Mycoplasma synoviae (MS) live vaccine candidate and presents studies of the safety and efficacy of the vaccine candidates. This example summarizes data from five animal trials (see Table 1). A MS challenge model was also developed.

TABLE 1
Summary of Experiments.

| | | | | Co-challenge | |
|---|---|---|---|---|---|
| Trial | | | Inoculation/ | (respiratory | |
| No. | | | Challenge | virus vaccine) | Notes |
| 1 | Safety I | Preliminary | Aerosol only | None | Weak |
| | | screening (pilot | (K1968 positive | | |
| | | study) | control) | | challenge |
| 2 | Efficacy I | Preliminary | Aerosol only | None | Weak |
| | | screening (pilot | (K1968) | | challenge |
| | | study) | | | |
| 3 | Safety II | | Intratracheal, | IBV (Mass) | Too strong |
| | | | Intraairsac, | | challenge |
| | | | Intrafootpad | | |
| | | | (K6677 positive | | |
| | | | control) | | |
| 4 | Efficacy II | | Intratracheal, | NDV (B1B1) | Too strong |
| | | | Intraairsac, | | |
| | | | Intrafootpad | | challenge |
| | | | (K6677) | | |
| 5 | Efficacy III | Comparison to | Aerosol, | NDV (B1B1) | Co-challenge |
| | | MS-H vaccine | Intrafootpad | | may be |
| | | strain | (K6677) | | unnecessarily |
| | | | | | complicate |
| | | | | | trials with |
| | | | | | K6677 |

Mycoplasma synoviae (MS) vaccine candidates were evaluated for safety and efficacy in five experiments. In Trials 1 and 2 the safety and efficacy of the candidates were evaluated, and although the challenge strategy resulted in very mild lesions and so needed improvement, it was evident that two of the candidates (K5885A and K4971B) showed promise as safe and potentially efficacious vaccines as they resulted in mild lesions after inoculation of naïve birds and significant reduction of colonization with the challenge strain (P<0.05). In Trials 3 and 4, the vaccine candidate K5885A was further evaluated for safety and efficacy using an approach that resulted in more severe MS challenge and lesions. K5885A inoculation (at 1000× the dose of the positive control strain) resulted in fewer (and less severe) air sac lesions and significantly less footpad lesions (P<0.05). There were also significantly less air sac and footpad lesions in birds vaccinated with K5885A following virulent challenge compared to non-vaccinated controls (P<0.05). In the final trial (Trial 5) the efficacy of K5885A was compared to the MS vaccine strain K3928 (MS-H) and protection from air sac lesions was comparable for both vaccines; but K5885A vaccination also significantly reduced footpad lesions, unlike K3928/MS-H (P<0.05). Although further research is necessary these preliminary studies indicate that K5885 is likely a safe and highly efficacious vaccine.

### Materials and Methods

MS strains and isolates. The vaccine candidates were selected from MS field isolates in the Mycoplasma culture depository at PDRC based on a case history where clinical disease was absent. K5885A was isolated from broiler breeder chickens in Arkansas in 2006; K4971B was isolated from commercial layer chickens in Georgia in 2000; K5805A was isolated from broiler breeder chickens in Alabama in 2005. The laboratory strain K1968 is a virulent MS strain the characteristics of which have already been described (Lockaby et al., 1998, Vet Pathol; 35:178-190); it was used as a control to evaluate the safety of the vaccine candidate strains in Trial 1 and as the challenge strain in Trial 2 to evaluate the efficacy of the vaccine candidates. In subsequent trials the isolate K6677 was used as the positive control/challenge strain. K6677 was isolated from broilers in Georgia in 2014; it is an unusually virulent MS strain. In Trial 5 the efficacy of the selected vaccine candidate (K5885A) was compared to the MS-H vaccine strain (K3928 in the PDRC repository); the characteristics of MS-H have been previously described (Morrow et al., 1998, Avian Diseases; 42:667-670; Markham et al., 1998, Avian Diseases; 42:677-681; Markham et al., 1998, Avian Diseases; 42:671-676; Markham et al., 1998, Avian Diseases; 42:682-689; and Noormohammadi et al., 2007, Avian Diseases; 51:550-554).

Inoculation and Challenge Procedures. For aerosol inoculation/challenge the MS isolates were administered using a commercial paint sprayer (Preval® Sprayer Division, Precision Valve Corporation, Yonkers, NY). Approximately 1 ml of actively growing culture was sprayed per bird. For eye drop, intra air sac, intra foot pad, and intra tracheal inoculation 100 μl of the isolates were administered to the site. The isolates were titrated (CCU/ml) at the time of inoculation using methods that have been previously described (Rodwell, A. W., and R. F. Whitcomb. Methods of Direct and Indirect Measurement of Mycoplasma Growth. In: Methods in Mycoplasmology. Volume I, Mycoplasma Characterization. S. Razin and J. G. Tully, eds. Academic Press, New York. pp 85-196. 1983). The titers of the isolates and the dose inoculated for all of the trials are summarized in Table 2.

TABLE 2
Summary of vaccine and challenge isolates titers and doses per bird.

| | | | Titer | Total Dose | Inoculation/ |
|---|---|---|---|---|---|
| Trial | | Vaccine/ | (CCU | per bird | Challenge |
| No. | | Challenge | per ml) | (CCUlog10) | Method |
| 1 | Safety I | K5885A | 2.9 × 10⁷ | 7.5 | 1 ml aerosol |
| | | K4971B | 3.6 × 10⁷ | 7.6 | |
| | | K5805A | 1.9 × 10⁸ | 8.3 | |
| | | K1968 | 1.9 × 10⁸ | 8.3 | |
| 2 | Efficacy I | K1968 | 4.7 × 10⁶ | 6.7 | |
| 3 | Safety II | K5885A | 2.8 × 10⁸ | 7.9 | 0.1 ml intratracheal + |
| | | K6677 | 1.4 × 10⁵ | 4.6 | 0.1 ml intraairsac + |
| 4 | Efficacy II | K6677 | 3.6 × 10⁶ | 6.0 | 0.1 ml intrafootpad |
| 5 | Efficacy | K5885A | 1.6 × 10⁷ | 6.2 | 0.1 ml eyedrop |
| | III | K3928/MS-H | 5.0 × 10⁵ | 4.7 | |
| | | K6677 | 8.0 × 10⁷ | 7.9 | 1 ml aerosol + |
| | | | 4.2 × 10⁷ | | 0.1 ml intrafootpad |

Serology. Sera in all trials were tested for the presence of MS antibodies with the serum plate agglutination (SPA) test using commercial antigen (Charles River Laboratories International, Inc., Wilmington, MA); the hemagglutination inhibition (HI) test, using antigen prepared from the WVU1853 strain and the enzyme-linked immunosorbent assay (ELISA) test, using a commercial kit (IDEXX, Westbrook, Maine).

The SPA and HI test procedures were as described by Ferguson-Noel et al. (Ferguson-Noel, N., and S. H. Kleven Mycoplasma species. In: A Laboratory Manual for the Isolation, Identification and Characterization of Avian Pathogens, Sixth ed. S. M. Williams, L. Dufour-Zavala, M. W. Jackwood, M. D. Lee, B. Lupiani, W. M. Reed, E. Spackman, and P. R. Woolcock, eds. American Association of Avian Pathologists. pp 63-70. 2016). An SPA score >1 was considered positive. An HI titer of >1:20 was considered positive. A geometric mean sample/positive (S/P) ratio of >0.5 on the ELISA test was considered positive.

Isolation and identification of mycoplasma. Cotton swabs from trachea, choanal cleft and air sacs were used for culture. They were inoculated in Frey's modified broth and agar and incubated at 37° C. Mycoplasma isolates were identified using direct immunofluorescence (10).

Real-Time Quantitative PCR. Real-time quantitative PCR (qPCR) was carried out using the procedure described by Raviv (Raviv and Kleven, 2009, Avian Diseases; 53:103-107). At necropsy the larynxes of individual birds were collected in 4 ml sterile PBS. Genomic DNA was extracted from 100 μl of the laryngeal wash or choanal cleft swabs using the Mag-Bind® Blood and Tissue DNA HDQ 96 kit (Omega Bio-tek, Inc., Norcross, GA) on the MagMAX™ Express-96 Magnetic Particle Processors (Thermo Fisher Scientific) following the manufacturer's recommendations. Real-time PCR was performed using an Applied Biosystems 7500 Fast Real-Time PCR System (Thermo Fisher Scientific) and a cycle threshold (Ct) value <39 was considered positive. To make the assays quantitative, plasmids were constructed containing the genome target (16S-23S rDNA ISR) as standard DNA controls. The procedures used in constructing the DNA controls and standard curves for quantitation have been described in detail elsewhere (Raviv et al., 2008, Vet Microbiol; 129:179-187).

Evaluation of lesions. Gross airsac and footpad lesions were scored on a scale of 0 to 4 using scoring systems described by Kleven (Kleven et al., 1972, Avian Diseases; 16:915-924; and Kleven et al., 1975, Avian Diseases; 19:126-135). Tracheal lesions were evaluated microscopically by measuring the width of the tracheal mucosa. A section of the upper third of the trachea (approximately 1 inch distal from the larynx) was fixed in 10% neutral formalin. The tracheal mucosa thickness was measured at four equidistant points on histological slides of cross sections of tracheas (Whithear, 1996, Rev Sci Tech; 15:1527-15).

Statistical analysis. The air sac lesion scores, footpad lesion scores, and SPA scores were analyzed using the Kruskal-Wallis Rank Sums test. The tracheal mucosa thickness, mean copy numbers (MCN) Log₁₀, s/p ratios, and HI titer Log₁₀ were analyzed using the Tukey-Kramer HSD test. Software from JMP® Statistics Made Visual (SAS Institute Inc., SAS Campus Drive, Cary, NC 27513). AP value <0.05 was considered significant.

### Safety I and Efficacy I

The aim of this portion of the study was preliminary screening of selected vaccines candidates for evidence of safety in naïve broiler chickens (Trial 1) and efficacy with respect to protection from tracheal and air sac lesions following challenge with a virulent MS isolate (K1968) (Trial 2).

### Experimental Design

Trial 1 (Safety I). One hundred and ten day-old commercial broiler-type chickens were acquired from a source known to be free of MS and Mycoplasma gallisepticum (MG) for Trials 1 and 2. They were housed in a floor pen (1.5×3 m²) with pine shavings litter in a naturally ventilated curtain-sided poultry house. At 14 days of age, they were randomly divided into 5 experimental groups and transferred to 5 colony houses (3×3 m²) with concrete floors and pine shavings litter. At this time, 10 chickens were randomly selected (2 per group) and tested by SPA, HI, ELISA; swabs of the choanal cleft were tested by culture and PCR to confirm that they were Mycoplasma negative.

At 21 days of age, 3 groups of 25 birds each were inoculated (via aerosol) with broth cultures of K5885A (2.9×10⁷ CCU/ml), K4971B (3.6×10⁷ CCU/ml) or K5805A (1.9×10⁸ CCU/ml). A fourth group of 15 birds was inoculated with K1968 (1.9×10⁸ CCU/ml) (controls). A negative control group of 10 chickens remained uninfected.

At 10 days post infection (DPI), ten birds from each of the MS-inoculated groups and 5 birds from the negative control group were euthanized and evaluated by air sac and lesion scoring. Samples were also collected for serology (SPA, HI, and ELISA), mycoplasma culture (choanal cleft/trachea and air sac swabs), and histopathology (tracheal sections). At 14 DPI, five birds from the MS-inoculated groups were euthanized and evaluated as described.

Trial 2 (Efficacy I). At 6 weeks post infection with the vaccine candidates, the remaining vaccinated birds from Trial 1 were inoculated (via aerosol) with broth culture of K1968 (4.7×10⁶ CCU/ml). Five birds were non-vaccinated controls and 5 birds were negative controls (not challenged). At 10 days post challenge (DPC) all of the birds were necropsied and evaluated as above.

### Results and Discussion

Trial 1. Safety I. The groups challenged with the K5885A and K5805A showed signs of seroconversion at 10 days post challenge (DPC), with 90% (9/10) and 100% (10/10) of birds reacting on the SPA test respectively. The lesions associated with MS infection in this trial were not severe and even infection with the positive control K1968, a pathogenic MS strain, did not result in severe air sac or tracheal lesions. As a result, (combined with the small numbers of birds in this pilot study), there were few significant differences in lesions associated with MS infection among the groups (P<0.05).

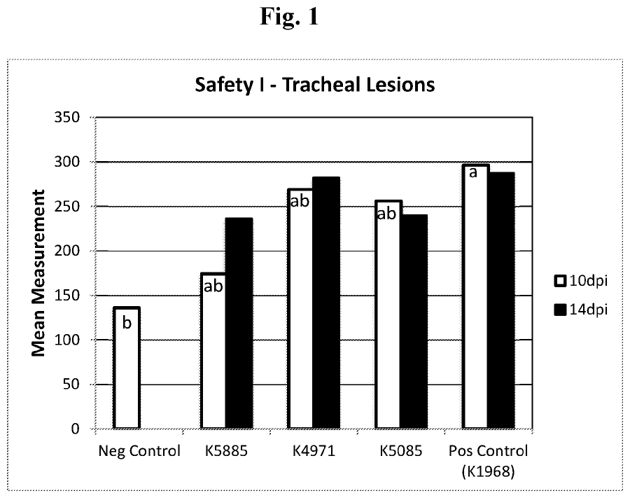

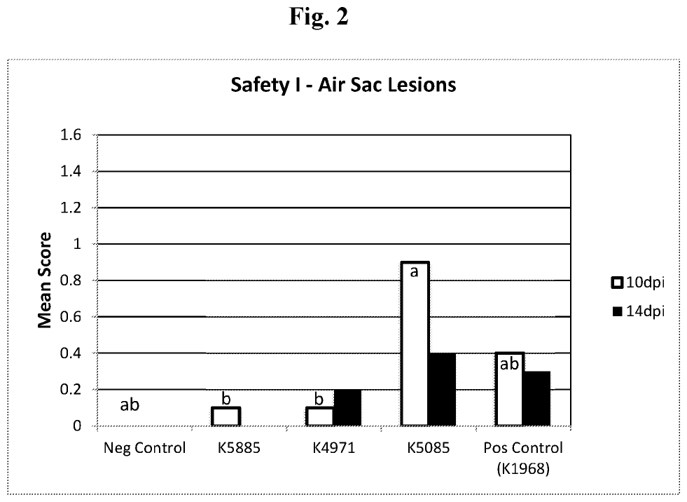

However, one of the MS candidates, K5805A, resulted in air sac lesions that were more severe than K1968 and significantly higher than K5885A and K4971B at 10 DPI (P<0.05). With respect to tracheal lesions, there were no significant differences in mean tracheal mucosal thickness among the MS-inoculated groups although the mean measurement for the K1968 group as significantly higher than the negative controls (P<0.05). The candidate K5885A resulted in the smallest increase in tracheal mucosal thickness at 10 and 14 DPI among the MS-infected groups. K5805A was eliminated from future studies due to the air sac lesions and lack of indication that the strain would produce a safe vaccine. These results are summarized in Table 3, and FIGS. 1 and 2.

TABLE 3
Trial 1 (Safety I). Serological response, lesion scores and MS isolation
from chickens at 10 and 14 DPI with K5885A, K4971B, K5805A or K1968^{A}.

| | | | | | | Tracheal | MS isolation | |
|---|---|---|---|---|---|---|---|---|
| | | | | | Air sac | mucosal | Choanal | Air |
| Infection | DPI | SPA | HI | ELISA | lesion score | thickness | Cleft | sacs |
| None | 10 | 0/5 ^{B} | 0/5 | 0/5 | 0/5 ^{F} | 136.1 ± 33.3^{Gb} | 0/5^{b} | 0/5^{d} |
| | | (0.0)^{C} | (0.0)^{D} | (0.0)^{E} | (0.0)^{ab} | | | |
| K5885A | 10 | 9/10 | 0/10 | 0/10 | 1/10 | 174.3 ± 45.8^{ab} | 10/10^{a} | 8/10^{ab} |
| | | (1.2)^{a} | (0.0) | (0.1) | (0.1)^{b} | | | |
| K4971B | 10 | 3/10 | 0/10 | 0/10 | 1/10 | 268.9 ± 174.1^{ab} | 10/10^{a} | 2/8^{cd} |
| | | (0.3)^{b} | (0.0) | (0.0) | (0.1)^{b} | | | |
| K5805A | 10 | 10/10 | 0/10 | 1/10 | 5/10 | 256.1 ± 51.4^{ab} | 10/10^{a} | 10/10^{a} |
| | | (1.1)^{a} | (0.0) | (0.2) | (0.9)^{a} | | | |
| K1968 | 10 | 3/10 | 0/10 | 1/10 | 4/10 | 296.3 ± 102.1^{a} | 10/10^{a} | 7/10^{bc} |
| | | (0.3)^{b} | (0.0) | (0.2) | (0.4)^{ab} | | | |
| K5885A | 14 | 4/5 | 0/5 | 0/5 | 0/5 | 235.7 ± 45.5^{G} | 5/5 | 4/5^{a} |
| | | (1.2) | (0.0) | (0.1) | (0.0) | | | |
| K4971B | 14 | 3/5 | 0/5 | 0/5 | 1/5 | 281.8 ± 94.2 | 5/5 | 0/5^{b} |
| | | (0.6) | (0.0) | (0.1) | (0.2) | | | |
| K5805A | 14 | 2/5 | 0/5 | 0/5 | 2/5 | 239.5 ± 66.3 | 5/5 | 2/5^{ab} |
| | | (0.8) | (0.0) | (0.1) | (0.4) | | | |
| K1968 | 14 | 1/3 | 0/3 | 0/3 | 1/3 | 287.0 ± 192.2 | 3/3 | 1/3^{ab} |
| | | (0.3) | (0.0) | (0.2) | (0.3) | | | |

^{A}Values within a column at a specific time point (10 or 14 DPI) with a different lower case superscript are significantly different (P ≤ 0.05)
^{B} No. of positive samples/No. of tested samples (SPA: ≥1, HI: ≥40, and ELISA: ≥0.5, Air sac score ≥1)
^{C}Mean agglutination grade (from 0 to 4).
^{D}Mean titer log10
^{E}Mean sample/positive ratio
^{F} Macroscopically scored from 0 to 4
^{G}Mean thickness (μm) ± SD

Trial 2. Efficacy I. As with the safety trial, there were no severe lesions associated with MS infection following challenge with K1968 by aerosol. There were no significant differences among the groups (vaccinated or negative or positive controls) with respect to air sac or tracheal lesions so few conclusions can be drawn from the data with respect to efficacy (P<0.05). However, there were significantly fewer MS isolations from the air sacs in the vaccinated groups, indicating that vaccination may have prevented systemic infection with the challenge strain (P<0.05). These results are summarized in Table 4.

TABLE 4
Trial 2 (Efficacy I). Serological response and lesion scores of vaccinated
and non-vaccinated chickens 10 days post challenge with K1968^{A}.

| | | | | Air sac | Tracheal | | |
|---|---|---|---|---|---|---|---|
| | | | | lesion | mucosal | MS isolation | |
| Vaccine | Challenge | SPA | HI | score | thickness | Trachea | Air sacs |
| None | None | 0/5^{B} | 0/5 | 0/5^{E} | 161.0 ± 52.6^{F} | 0/5^{b} | 0/5^{ab} |
| | | (0.0)^{cC} | (0.0)^{bD} | (0.0)^{E} | | | |
| K5885A | Yes | 7/8 | 2/8 | 3/8 | 217.9 ± 71.6 | 8/8^{a} | 0/8^{b} |
| | | (2.6)^{a} | (1.1)^{a} | (0.5) | | | |
| K4971B | Yes | 10/10 | 2/10 | 1/10 | 238.2 ± 88.3 | 10/10^{a} | 0/10^{b} |
| | | (2.4)^{a} | (0.5)^{ab} | (0.1) | | | |
| None | Yes | 5/5 | 0/5 | 2/5 | 257.3 ± 82.5 | 4/4^{a} | 2/5^{a} |
| | | (1.2)^{b} | (0.0)^{b} | (0.6) | | | |

^{A}Values within a column with a different lower case superscript are significantly different (P ≤ 0.05)
^{B}No. of positive samples/No. of tested samples (SPA: ≥1, HI: ≥40, and ELISA: ≥0.5, Air sac score ≥1)
^{C}Mean agglutination grade (from 0 to 4)
^{D}Mean titer log10
^{E}Macroscopically scored from 0 to 4
^{F}Mean thickness (μm)

Following this trial, the challenge method was optimized to result in more severe lesions using a recently isolated positive control strain (K6677). Changes in inoculation methods (intra tracheal, intra air sac and intra footpad) also increased the severity of the challenge. Respiratory virus vaccines (Newcastle Disease Virus (NDV) and Infectious Bronchitis Virus (IBV)) that are routinely used in poultry production were also included. K4971B was eliminated from future trials due to difficulties with in vitro growth of this strain.

### Safety II and Efficacy II

The aim of this portion of the study was to investigate the safety of the selected vaccine candidate following a high dose inoculation by several invasive routes and co-administration of a respiratory virus vaccine (Trial 3). The protection of vaccinated birds from tracheal, air sac and foot pad lesions were investigated following challenge with a virulent MS isolate (K6677) (Trial 4).

### Experimental Design

Trial 3 (Safety II). One hundred day-old commercial broiler-type chickens were acquired from a source known to be free of MS and MG for Trials 3 and 4. They were housed in a floor pen (1.5×3 m²) with pine shavings litter in naturally ventilated curtain sided poultry house. At 21 days of age, they were randomly divided into 6 experimental groups and transferred to 6 floor pens. At this time, 20 chickens were randomly selected and tested by MG and MS SPA, HI, ELISA; swabs of the choanal cleft were tested by culture and PCR to confirm that they were Mycoplasma, NDV and IBV negative.

Also, at 21 days of age, 30 birds were inoculated with the vaccine candidate K5885A (2.8×10⁸ CCU/ml) (via intratracheal, intrafootpad and intra airsac routes) and NDV vaccine (B1B1; via eyedrop route). Fifteen birds were inoculated with K6677 (1.4×10⁵ CCU/ml) and NDV vaccine (positive controls); and 10 birds were inoculated with NDV only. An additional 10 birds were not inoculated and served as negative controls.

At 14 DPI, fifteen birds from each of the MS-inoculated groups and 10 birds each from the NDV-only and negative control groups were euthanized and evaluated by air sac and lesion scoring. Samples were also collected for serology (SPA, HI, and ELISA), mycoplasma culture (air sac swabs), histopathology (tracheal sections), and quantitative real-time PCR (tracheal sections).

Trial 4 (Efficacy II). At 6 weeks post infection with the vaccine candidate, 10 birds remaining from Trial 1 were selected and tested by serology and PCR. At this time, the K5885A-vaccinated chickens (n=17) were also inoculated (via aerosol) with broth culture of K6677 (3.6×10⁶ CCU/ml) and IBV vaccine. Eighteen birds were nonvaccinated controls, 11 birds were only vaccinated with IBV, and 9 birds were negative controls (not challenged or vaccinated). At 14 DPC, all of the birds were necropsied and evaluated as above.

### Results and Discussion

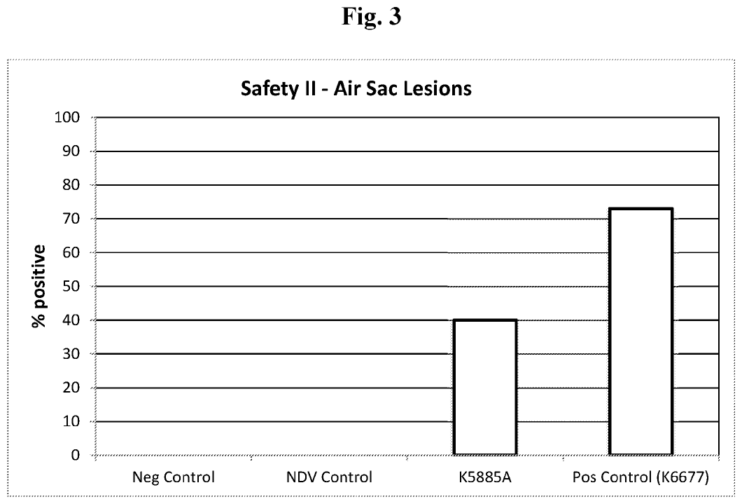

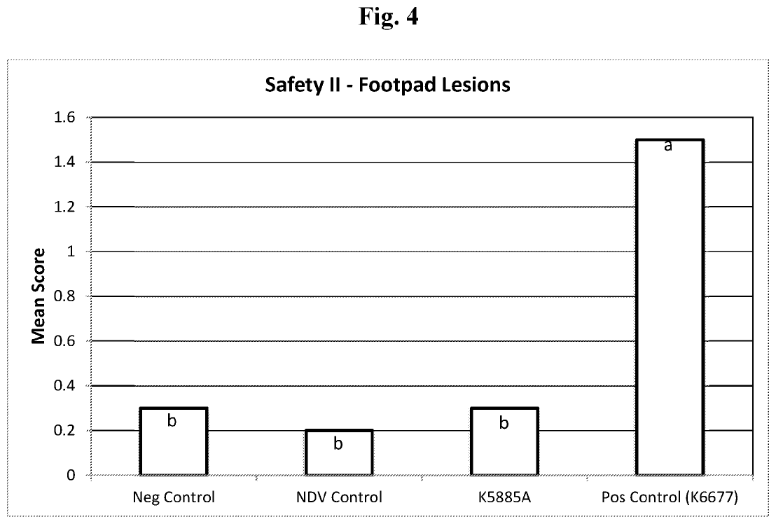

Trial 3. Safety II. There was evidence of seroconversion at 14 days post infection with both K5885A and K6677 on the SPA and ELISA tests (see Table 5). The challenge in this trial was much stronger as the birds were infected via intra tracheal, intra air sac and intra footpad inoculation. This challenge resulted in air sac lesions in both MS infected groups, although there were fewer affected birds in the K5885A group (40% (6 of 15) vs. 73% (11 of 15). There were also significantly fewer birds affected with footpad lesions (P<0.05). It should be noted that the titer of K5885A was much higher than the titer of the control strain (2.8×10⁸ CCU/ml for K5885A vs. 1.4×10⁵ CCU/ml for K6677). Tracheal mucosal measurement comparisons are pending. The isolate K5885A was detected in all of the inoculated birds and replicated at levels comparable to the positive control K6677 as indicated by qPCR and MS isolation. These results are summarized in Table 6 and FIGS. 3 and 4.

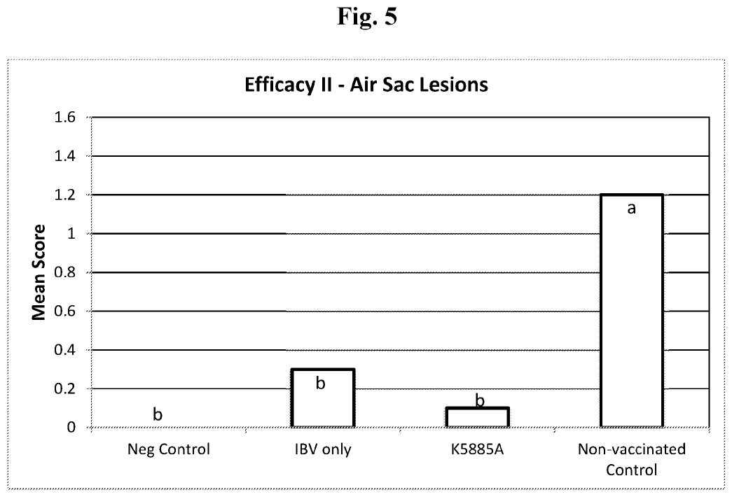

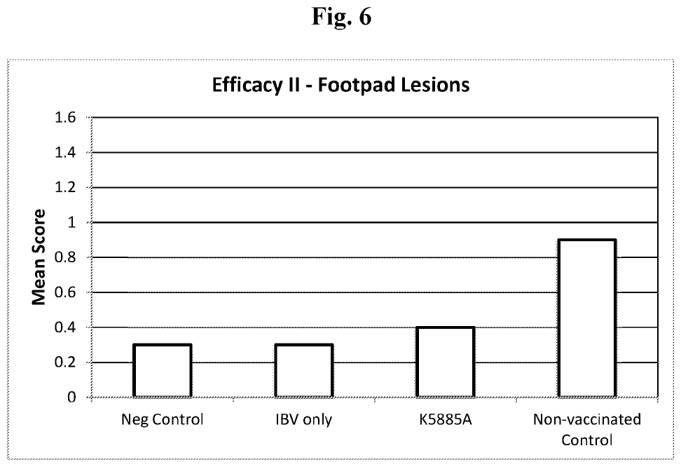

Trial 4. Efficacy II. At 6 weeks after vaccination (at the time of challenge), the serology and PCR results indicated that the non-inoculated birds remained negative for MS. The vaccinated group did seroconvert of all of the serological tests and they were also MS PCR positive at that time. At 14 DPC seroconversion was similar for both the vaccinated group and the non-vaccinated but challenged group, although the mean ELISA s/p ratio was significantly lower for the vaccinated group (see Table 7) (P<0.05). There were significantly less airsacculitis and footpad lesions in birds that had been vaccinated with K5885A despite the strong challenge with K6677 (P<0.05). There were also significantly fewer isolations of MS from the air sacs and lower MCN log 10 in the trachea indicating that there was less replication and systemic infection with the MS challenge strain (despite direct inoculation of these sites) (P<0.05). These results are summarized in Table 8 and FIGS. 5 and 6.

TABLE 5
Trial 3 (Safety II). Serological response of
chickens at 14 DPI with K5885A or K6677^{A}.

| MS | NDV | | | | | | |
|---|---|---|---|---|---|---|---|
| Infection | Vaccination | SPA | | HI | | ELISA | |
| None | None | 0/10^{B} | (0.0)^{bC} | 0/10 | (0.0)^{D} | 0/10 | (0.0)^{bE} |
| None | Yes | 0/11 | (0.0)^{b} | 0/11 | (0.0) | 0/11 | (0.0)^{b} |
| K5885A | Yes | 11/15 | (1.3)^{a} | 0/15 | (0.0) | 7/15 | (0.8)^{ab} |
| K6677 | Yes | 11/15 | (0.8)^{a} | 0/15 | (0.0) | 5/15 | (1.5)^{a} |

^{A}Values within a column at a specific time point with a different lower case superscript are significantly different (P ≤ 0.05)
^{B}No. of positive samples/No. of tested samples (SPA: ≥1, HI: ≥20, and ELISA: ≥0.5)
^{C}Mean agglutination grade (from 0 to 4).
^{D}Mean titer log10
^{E}Mean s/p ratio

TABLE 6
Trial 3 (Safety II). Lesion scores and MS isolation
from chickens at 14 DPI with K5885A or K6677^{A}

| MS | NDV | Air sac lesion | | Footpad lesion | | MS isolation | qPCR |
|---|---|---|---|---|---|---|---|
| Infection | Vaccination | score | | score | | (Air sacs) | (Trachea) |
| None | None | 0/10^{B} | (0.0)^{bC} | 3/10^{C} | (0.3)^{bC} | 0/10^{b} | 0.00^{bD} |
| None | Yes | 0/11 | (0.0)^{b} | 2/11 | (0.2)^{b} | 0/11^{b} | 0.0^{b} |
| K5885A | Yes | 6/15 | (0.6)^{ab} | 3/15 | (0.3)^{b} | 14/15^{a} | 3.0 ± 0.6^{a} |
| K6677 | Yes | 11/15 | (1.0)^{a} | 10/15 | (1.5)^{a} | 11/15^{a} | 2.8 ± 0.7^{a} |

^{A}Values within a column with a different lower case superscript are significantly different (P ≤ 0.05)
^{B}No. of positive samples/No. of tested samples (Air sac score ≥ 1; Footpad score ≥ 1)
^{C}Macroscopically scored from 0 to 4
^{D}Mean (genome) copy number log 10

TABLE 7
Trial 4 (Efficacy II). Serological response of vaccinated and
non-vaccinated chickens 14 days post challenge with K6677^{A}.

| MS | IBV | MS | | | | | | |
|---|---|---|---|---|---|---|---|---|
| Vaccine | Vaccination | Challenge | SPA | | HI | | ELISA | |
| None | None | None | 0/9^{B} | (0.0)^{bC} | 0/9 | (0.0)^{bD} | 0/9 | (0.2)^{cE} |
| None | Yes | None | 0/11 | (0.0)^{b} | 0/11 | (0.0)^{b} | 2/11 | (0.5)^{c} |
| K5885A | Yes | Yes | 16/16 | (2.3)^{a} | 17/17 | (2.4)^{a} | 16/16 | (4.3)^{b} |
| None | Yes | Yes | 18/18 | (2.1)^{a} | 17/18 | (2.1)^{a} | 18/18 | (9.6)^{a} |

^{A}Values within a column with a different lower case superscript are significantly different (P ≤ 0.05)
^{B}No. of positive samples/No. of tested samples (SPA: ≥1, HI: ≥40, and ELISA: ≥0.5)
^{C}Mean agglutination grade (from 0 to 4).
^{D}Mean titer log10
^{E}Mean s/p ratio

TABLE 8
Trial 4 (Efficacy II). Lesion scores of vaccinated and non-
vaccinated chickens 14 days post challenge with K6677^{A}.

| MS | IBV | MS | Air sac | Footpad | MS Isolation | qPCR |
|---|---|---|---|---|---|---|
| Vaccine | Vaccination | Challenge | lesion score | lesion score | (Air Sacs) | (Trachea) |
| None | None | None | 0/9^{B} | 3/9 | 0/9^{b} | 0/9 |
| | | | (0.0)^{bC} | (0.3)^{C} | | (0.0)^{cD} |
| None | Yes | None | 3/11 | 3/11 | 0/11^{b} | 0/11 |
| | | | (0.3)^{b} | (0.3) | | (0.0)^{c} |
| K5885A | Yes | Yes | 1/17 | 4/17 | 2/17^{b} | 15/17 |
| | | | (0.1)^{b} | (0.4) | | (2.1 ± 1.4)^{b} |
| None | Yes | Yes | 10/18 | 11/18 | 13/18^{a} | 18/18 |
| | | | (1.2)^{a} | (0.9) | | (3.4 ± 0.6)^{a} |

^{A}Values within a column with a different lower case superscript are significantly different (P ≤ 0.05)
^{B}No. of positive samples/No. of tested samples (Air sac score ≥1, Foot pad score ≥1)
^{C}Macroscopically scored from 0 to 4
^{D}Mean (genome) copy number log 10

### Efficacy III

The aim of this portion of the study was to compare the efficacy of the selected vaccine candidate to commercial MS vaccine strain (MS-H) with respect to tracheal, air sac and foot pad lesions following challenge with a virulent MS isolate (K6677). The dose and route of vaccination were more comparable to field MS vaccination practices as the vaccines were administered via eye drop at 3 weeks of age.

Experimental Design: One hundred day-old commercial broiler-type chickens were acquired from a source known to be free of MS and MG. They were housed in a colony house (3×3 m²) with concrete floors and pine shavings litter. At 21 days of age, they were randomly divided into 5 experimental groups and transferred to 5 new colony houses (3×3 m²) with concrete floors and pine shavings litter. At this time 15 chickens were randomly selected and tested by SPA and HI; swabs of the choanal cleft were tested by culture and PCR to confirm that they were Mycoplasma and NDV negative. Also, at 21 days of age, 20 birds were inoculated with the vaccine candidate K5885A (1.6×10⁷ CCU/ml) and 20 birds were inoculated MS-H vaccine strain (5.0×10⁵ CCU/ml) (both via eye drop).

At 5 weeks post vaccination the birds were challenged with K6677 (8.0×10⁷ CCU/ml) via aerosol and NDV vaccine (B1B1) via eye drop. Twenty naïve birds were inoculated with NDV vaccine only and 20 birds remained uninfected as negative controls. One week post aerosol challenge, the challenged groups were also inoculated with K6677 (4.2 10⁷ CCU/ml) via footpad. At 14 DPC the birds were euthanized and evaluated as previously described.

### Results and Discussion

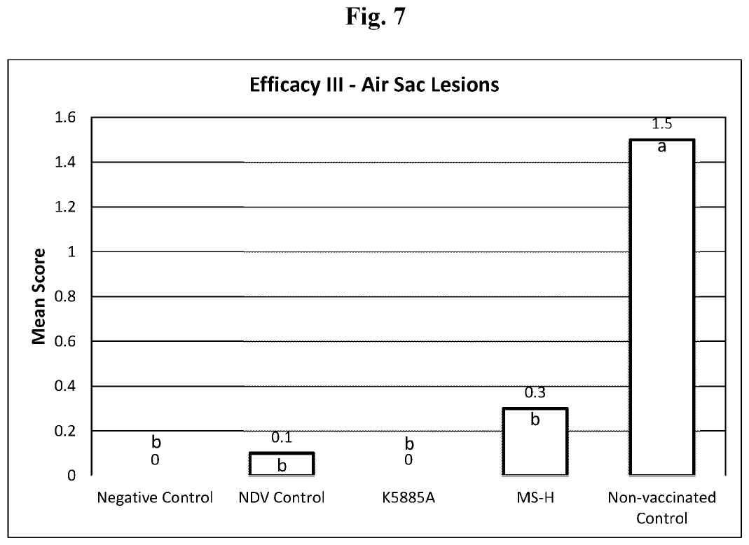

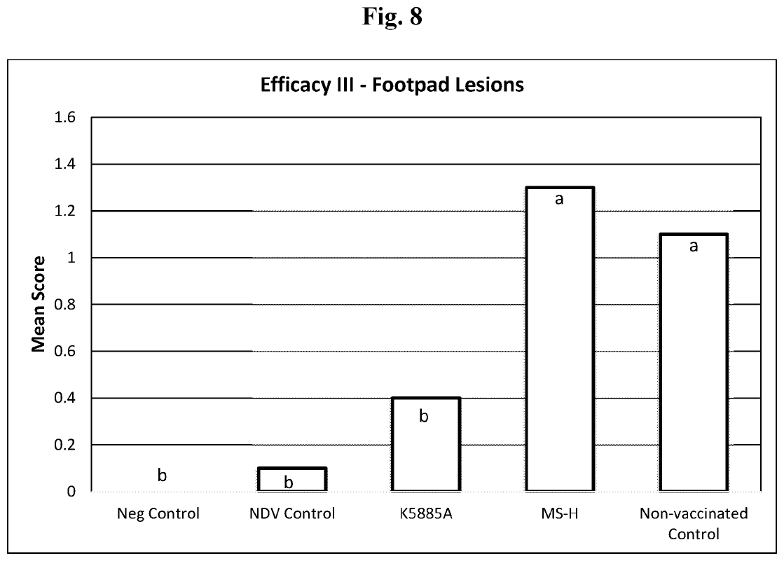

Seroconversion following challenge was similar for both vaccinated groups in this trial, although there were slightly lower titers on the HI tests and significantly lower titers on the ELISA test in the group vaccinated with K5885A (see Table 9) (P<0.05). There was significantly less airsacculitis in birds that had been vaccinated with K5885A and MS-H (P<0.05) in this trial; however only vaccination with K5885A resulted in a significant reduction in footpad lesions (P<0.05). Although there were significantly fewer isolations of MS from the air sacs from birds vaccinated with wither K5885A or MS-H, only MS-H resulted in less replication of MS in the trachea with significantly lower MCN log 10 (P<0.05). These results are summarized in Table 10 and FIGS. 7 and 8

TABLE 9
Trial 5 (Efficacy III). Serological response and lesion scores of vaccinated
and non-vaccinated chickens 14 days post challenge with K6677^{A}.

| | NDV | MS | | | | | | |
|---|---|---|---|---|---|---|---|---|
| Vaccine | Vaccination | Challenge | SPA | | HI | | ELISA | |
| None | None | None | 0/20^{B} | (0.0)^{bC} | 0/20 | (0.0)^{cD} | 0/20 | (0.0)^{dE} |
| None | Yes | None | 0/20 | (0.0)^{b} | 0/20 | (0.0)^{c} | 0/20 | (0.1)^{d} |
| K5885A | Yes | Yes | 15/15 | (3.9)^{a} | 15/15 | (1.5)^{ab} | 15/15 | (1.8)^{c} |
| MS-H | Yes | Yes | 18/18 | (3.7)^{a} | 17/18 | (1.5)^{a} | 18/18 | (3.5)^{b} |
| None | Yes | Yes | 20/20 | (3.5)^{a} | 14/20 | (1.3)^{b} | 20/20 | (5.1)^{a} |

^{A}Values within a column with a different lower case superscript are significantly different (P ≤ 0.05)
^{B}No. of positive samples/No. of tested samples (SPA: ≥1, HI: ≥20, and ELISA: ≥0.5)
^{C}Mean agglutination grade (from 0 to 4)
^{D}Mean titer log10
^{E}Mean s/p ratio

TABLE 10
Trial 5 (Efficacy III). Serological response and lesion scores of vaccinated
and non-vaccinated chickens 14 days post challenge with K6677^{A}.

| | | | | Footpad | MS | Tracheal | |
|---|---|---|---|---|---|---|---|
| | NDV | MS | Air sac | lesion | Isolation | mucosal | qPCR |
| Vaccine | Vaccination | Challenge | lesion score | score | (Air sacs) | thickness | (Trachea) |
| None | None | None | 0/20^{B} | 0/20 | 0/11^{c} | 171.1 ± 38.0^{bD} | 0/20 |
| | | | (0.0)^{bC} | (0.0)^{bC} | | | (0.0)^{cD} |
| None | Yes | None | 1/20 | 1/20 | 0/20^{c} | 207.8 ± 58.4^{ab} | 0/20 |
| | | | (0.1)^{b} | (0.1)^{b} | | | (0.0)^{c} |
| K5885A | Yes | Yes | 0/20 | 7/20 | 9/18^{b} | 243.4 ± 72.2^{a} | 20/20 |
| | | | (0.0)^{b} | (0.4)^{b} | | | (3.8 ± 0.7)^{a} |
| MS-H | Yes | Yes | 3/19 | 17/19 | 8/16^{b} | 238.0 ± 52.2^{a} | 20/20 |
| | | | (0.3)^{b} | (1.3)^{a} | | | (2.8 ± 1.1)^{b} |
| None | Yes | Yes | 13/20 | 16/20 | 15/16^{a} | 250.2 ± 57.8^{a} | 20/20 |
| | | | (1.5)^{a} | (1.1)^{a} | | | (3.5 ± 0.6)^{a} |

^{A}Values within a column with a different lower case superscript are significantly different (P ≤ 0.05)
^{B}No. of positive samples/No. of tested samples (Air sac score ≥1; Footpad score ≥1)
^{C}Macroscopically scored from 0 to 4
^{D}Mean thickness for the group ± SD (μm)
^{E}Mean (genome) copy number log 10

It should be noted that the titer of the MS-H vaccine was lower than the titer of the K5885A vaccine (1.6×10⁷ CCU/ml for K5885A vs. 5.0×10⁵ CCU/ml for MS-H strain) and that the commercial vaccine was not used in this study; a higher titer as well as the commercial product may affect the efficacy. Strain-specific PCR protocols (currently under development) will be able to differentiate between replication and isolation of the vaccine strain compared to the challenge strain and so allow more useful analysis of these data.

This example demonstrates that K5885A is an MS isolate of low virulence and vaccination with this isolate results in protection from MS associated disease and to some extent subsequent MS infection with challenge strains. K5885A is therefore a safe and efficacious vaccine candidate.

### Example 2

Mycoplasma synoviae Challenge Model to Evaluate the Impact of Vaccination on Oviduct Colonization

To evaluate the efficacy of a Mycoplasma synoviae vaccine candidate in layer-type chickens in egg production, groups of chickens were vaccinated with 5.5 CCU log 10 K5885 by eye drop at 5 weeks of age and then challenged with two virulent M. synoviae strains (K6677 and WVU 1853) at 23 weeks of age on two sequential days by aerosol and foot pad inoculation. Groups of chickens were euthanized and examined for airsacculitis, footpad lesions, and ovarian regression at 2, 3, 4, 5 and 6 WPC. Eggshell strength and egg abnormalities were recorded, and eggs were cultured for M. synoviae pre and post challenge. Vaccination with K5885 resulted in significant protection from airsacculitis, footpad lesions (synovitis) and ovarian regression after challenge with either K6677 or WVU 1853 compared to the non-vaccinated groups that were challenged (P<0.05). There was also a significant reduction in K6677 colonization (as indicated by qPCR mean (genome) copy numbers log 10 from the trachea) in the vaccinated group compared to the non-vaccinated control group (P<0.05). A significant decline in egg production was observed from 2 to 5 weeks post challenge in the non-vaccinated groups that were challenged with either K6677 or WVU 1853 but not in the vaccinated groups (P<0.05). There were no significant differences in eggshell strength, egg abnormalities, colonization of the oviduct or isolation of M. synoviae from eggs among the groups during the study. These results demonstrate the potential the of K5885 vaccine candidate preventing clinical signs and egg production losses associated M. synoviae infection.

### INTRODUCTION

Mycoplasma synoviae (MS) infection is an important poultry disease that may cause tracheitis, airsacculitis, synovitis, and some negative reproductive effects, including eggshell apex abnormalities and reduced egg production in poultry, although subclinical infections of the respiratory tract seem predominant (Feberwee et al., 2009, Avian Pathology; 38(2):187-187; Gole et al., 2012, Preventive Veterinary Medicine; 106(1):75-78; Kleven et al., 1975, Avian Dis; 19(1):126-135; and Landman et al., 2004, Avian Pathology; 33(2):210-215). The clinical presentation of MS infection is be related to the MS strain, where MS isolates from air sac lesions induce airsacculitis, isolates from articular lesions induced joint pathology and more recently the emergence of the Dutch strains that cause eggshell apex abnormalities (EAA) and low egg production have now been encountered worldwide (Feberwee et al., 2009, Avian Pathology; 38(2):187-187; Ferguson-Noel et al., 2013, Mycoplasma synoviae infection. In D. E. Swayne, J. R. Glisson, L. R. McDougald, L. K. Nolan, D. L, Suarez, & V. Nair (Eds.), Diseases of Poultry (pp. 900-906). Wiley-Blackwell; and Landman, 2014, Avian Pathology; 43(1):2-8). EAA is characterized by altered shell surface, shell thinning, and cracks and breaks confined to a region up to approximately 2 cm from the apex of the egg (Feberwee et al., 2009, Avian Pathology; 38(2):187-187). The pathogenic process of MS infection involves attachment and colonization of the upper respiratory tract, and eventually the air sacs (resulting in airsacculitis); then spread to joints through hematogenous routes after colonization of the respiratory tract; and also the colonization of the oviduct, which has been hypothesized as a pre-requisite for the induction of eggshell abnormalities (Feberwee et al., 2009, Avian Pathology; 38(2):187-187; Ferguson-Noel et al., 2013, Mycoplasma synoviae infection. In D. E. Swayne, J. R. Glisson, L. R. McDougald, L. K. Nolan, D. L. Suarez, & V. Nair (Eds.), Diseases of Poultry (pp. 900-906). Wiley-Blackwell; and Kawakubo et al., 1980, J Comp Pathol; 90:457-467).

Transmission of MS can occur vertically, in ovo, or horizontally by direct contact or airborne spread and vaccination significantly reduced its shedding and horizontal transmission (Jones et al., 2006, Avian Dis; 50(1):88-91; and Noormohammadi et al., 2007, Avian Dis; 51(2):550-554). A live temperature-sensitive MS vaccine strain, MS-H, that was selected by mutagenesis of a virulent field isolate from Australia (Morrow et al., 1998, Avian Dis; 42(4):667-670) is used for the prevention of M. synoviae infections in many countries. Although its safety and efficacy in reducing airsacculitis have been established under laboratory and field conditions (Markham et al., 1998, Avian Dis; 42(4):677-681; Markham et al., 1998, Avian Dis; 42(4):671-676; and Markham et al., 1998, Avian Dis; 42(4): 682-689), there are no records of its efficacy in preventing MS footpad lesions (synovitis). Genome analysis of MS-H re-isolates recovered from vaccinated flocks showed the presence of some mutations similar to the parent strain sequence indicating that the MS-H strain reverted to the parent strain sequence (Kordafshari et al., 2020, Avian Pathology; 49(3):275-285; and Kordafshari et al., 2019, Veterinary Microbiology; 231:48-55). Concurrent reversions in ObgE, OppF, and GapdH proteins were associated with higher gross air sac lesion scores (and increased microscopic upper-tracheal mucosal thickness in chickens directly inoculated with the MS-H reisolates from the field following intratracheal administration of a virulent strain of infectious bronchitis virus (Klose et al., 2022, Front Microbiol; 13:1042212).

Described herein is a new MS vaccine (K5885) that was isolated from broiler breeder chickens in Arkansas in 2006, that significantly reduces loads of challenge MS in the upper respiratory tract, preventing airsacculitis and footpad lesions associated with MS infection.

### Objectives

The main objective of this study was to develop a Mycoplasma synoviae challenge model to evaluate the impact of K5885 vaccine candidate on oviduct colonization by two virulent MS challenge strains (K6677 and WVU 1853). The specific objectives included a bird trial to compare air sac lesions, footpad lesions, the quantity of the challenge strains in choanal cleft and trachea washes, egg production rate, and egg abnormalities in chickens vaccinated chickens with K5885 before challenged with two MS wild strains (K6677 and WVU 1853) and the non-vaccinated.

### Materials and Methods

MS isolates. The vaccine candidate (K5885A) was isolated from broiler breeder chickens in Arkansas in 2006 and selected from the Mycoplasma culture depository at the Poultry Diagnostic and Research Center (PDRC), University of Georgia, Athens. Isolates K6677 and WVU 1853 were used as the positive control/challenge strains. K6677 was isolated from broilers in Georgia in 2014; it is an unusually virulent MS strain and WVU 1853 is the M. synoviae type strain isolated in the USA (Zhu et al., 2018).

Vaccination and Challenge Procedures. Each vaccinated chicken received a dose of 5.5 CCU log 10 in 30 μl placed in the left eye. For aerosol challenge, the MS isolates (K6677 and WVU 1853) were administered using a commercial paint sprayer (PREVAL® Sprayer Division, Precision Valve Corporation, Yonkers, NY). Approximately 1 ml of actively growing culture was sprayed per chicken. For intra-foot pad inoculation, 100 μl of the isolates were administered to the site. The isolates were titrated (CCU/ml) at the time of inoculation using methods that have been previously described by Rodwell and Whitcomb, 1983. The titers of the MS isolates and dose inoculated for the experiment are summarized in Table 11.

Evaluation of lesions. Gross air sac and footpad lesions were scored on a scale of 0 to 4 using scoring systems previously described (Kleven et al., 1975, Avian Dis; 19(1):126-135). Ovarian regression was evaluated by gross examination; ovaries were scored as immature (juvenile), normal (multiple follicles in various stages of development) or regressed (multiple atretic follicles that were flaccid and/or discolored) (Ferguson-Noel et al., 2012, Avian Dis: 56(2):272-275.

Egg strength measurement. Eggs were collected two weeks pre-challenge and for six weeks post challenge for the egg strength test. Egg strength was determined by the shell-breaking method using an Egg force reader (Orka Technology Ltd, Manchester, England).

Serology. Sera were tested for the presence of MS antibodies with the serum plate agglutination (SPA) test using commercial antigen (Charles River Laboratories International, Inc., Wilmington, MA); the hemagglutination inhibition (HI) test, using antigen prepared from the WVU 18531853 strain and the enzyme-linked immunosorbent assay (ELISA) test, using a commercial kit (IDEXX, Westbrook, Maine). The SPA and HI test procedures were as described by Ferguson-Noel et al. 2016. A SPA score >1 was considered positive. An HI titer of >1:20 was considered positive. A geometric mean sample/positive (S/P) ratio of >0.5 on the ELISA test was considered positive.

Isolation and identification of mycoplasma. Egg yolks, choanal cleft swabs and oviduct swabs were used for culture. Eggs were collected and incubated for six days on alternating days, starting from 2 days post challenge to 6 WPC. Egg yolk from incubated eggs were pooled (3 in 1) for MS culture. Swabs and egg yolks were inoculated in Frey's modified broth and agar and incubated at 37° C. Mycoplasma isolates were identified using direct immunofluorescence (Ferguson-Noel et al. 2016).

MS Real-Time Quantitative PCR. Real-time quantitative PCR (qPCR) was carried out using the procedure described by Raviv and Kleven, 2009. At necropsy, a section the upper trachea of individual chickens was collected in 9 ml sterile PBS as well as a swab of the oviduct. Genomic DNA was extracted from 200 μl of the upper trachea wash or oviduct swabs using the MAG-BIND® Blood and Tissue DNA HDQ 96 kit (Omega Bio-tek, Inc., Norcross, GA) on the MagMAX™ Express-96 Magnetic Particle Processors (Thermo Fisher Scientific) following the manufacturer's recommendations. Real-time PCR was performed using an Applied Biosystems 7500 Fast Real-Time PCR System (Thermo Fisher Scientific) and a cycle threshold (Ct) value <39 was considered positive. To make the assays quantitative, plasmids were constructed containing the genome target (16S-23S rDNA ISR) as standard DNA controls. The procedures used in constructing the DNA controls and standard curves for quantitation have been described in detail elsewhere (Raviv et al., 2008, Vet Microbiol; 129(1-2):179-187).

Strain Specific Real time PCR. Comparative genomics of the three MS strains in this study—K5885 (vaccine candidate) and K6677 (challenge strain) was used to identify targets and develop quantitative PCR protocols for the specific detection of these strains. The strain specific real-time PCR protocols were performed on samples prepared as described above using an Applied Biosystems 7500 Fast Real-Time PCR System (Thermo Fisher Scientific) and a cycle threshold (Ct) value <39 was considered positive. To make the assays quantitative, plasmids were constructed containing the specific genome targets as standard DNA controls as described previously (Raviv et al., 2008, Vet Microbiol; 129(1-2):179-187).

Chickens and Experimental Design. Three hundred and eighteen specific pathogen free (SPF) chicks were acquired from Charles River SPAFAS (Wilmington, DE) at four weeks of age (WOA). Chicks were randomly selected and placed into six groups: non-vaccinated non-challenged (NV/NC), non-vaccinated K6677-challenged (NV/C-K), non-vaccinated WVU 1853-challenged (NV/C-W), vaccinated K6677-challenged (V/C-K), vaccinated WVU 1853-challenged (V/C-W), and vaccinated non-challenged (V/NC). 60 chicks were randomly selected and place into each of NV/NC, NV/C-K, NV/C-W, V/C-K, and V/C-W groups while 18 chicks were in the V/NC group. The chickens were housed in floor pens with pine shavings litters in naturally ventilated curtain-sided poultry houses. At 5 weeks of age (WOA), 30 chickens were screened to confirm that the chickens were negative for avian Mycoplasma spp. by serology (SPA, HI, and ELISA) and culture of choanal cleft swabs and qPCR of tracheal washes. At 6 WOA, all chicks in V/C-K, V/C-W, and V/NC groups were vaccinated via an intra-ocular route with K5885 (MS vaccine candidate). At 4 weeks post vaccination (WPV) and 15 WPV, 30 chickens (15 non-vaccinated and 15 vaccinated) were randomly selected to confirm vaccination and negative status. At 2 weeks pre-challenge and at 3 to 6 weeks post challenge (WPC) all eggs were collected for eggshell strength testing on alternate days. At 23 WOA, when egg production was approximately 80% in all the groups, hens were challenged on two sequential days (aerosol inoculation only on the first day, while aerosol and intra foot pad inoculation on the second day). Chickens in NV/C-K and V/C-K groups were challenged with K6677 while hens in NV/C-W and V/C-W groups were challenged with WVU 1853 (challenge doses per group are summarized in Table 11). At 2, 3, 4, 5 and 6 WPC, 12 hens from all groups, except V/NC, were randomly selected and euthanized. In V/NC group, 6 hens selected and euthanized at 2, 4 and 6 WPC. All eggs were collected, on alternate days, starting from 2 days post challenge to the end of the study, and incubated for 6 days before they were cultured for MS.

The experimental design and procedures are summarized in Table 12. Throughout the study, chickens were provided with feed and water ad libitum, and euthanized by cervical dislocation and carbon dioxide according to the animal care and use policies of the University of Georgia, Athens, GA.

Statistical analysis. The air sac lesion scores, footpad lesion scores, HI titer Log 10, ELISA s/p ratios, SPA mean agglutination grade and eggshell strength were analyzed using the 2-way ANOVA with Turkey's multiple comparison test. For number ovarian regression observed per group, paired T-test was used to compare values between two different groups. The weekly egg production rate percentage and MS mean copy numbers (MCN) tracheal washes and oviduct swabs were analyzed using the 2-way ANOVA (Turkey's multiple comparison test) while the tracheal K6677 mean copy numbers (MCN) were analyzed using the 2-way ANOVA (Šidák's multiple comparison test). All statistical analyses were done using GraphPad Prism version 9.5.1 for macOS, GraphPad Software, www.graphpad.com”. A P value <0.05 was considered significant.

### Results

Pre-vaccination and Pre-Challenge Testing. The chickens tested for MS and MG pre-vaccination (5 WOA) were negative by culture, qPCR, and serology using SPA, HI, and ELISA. At 10 WOA (4 weeks post vaccination and pre-challenge) the samples from the non-vaccinated groups were all negative while the samples from the vaccinated groups were all MS positive by SPA, ELISA, culture, and PCR (4.0±0.4 MCN log 10). Only one (6.7%) of the samples from the vaccinated group was positive by HI at that time with a titer of 1.30 log 10. At 21 WOA (15 weeks post vaccination and pre-challenge) the chickens from the vaccinated groups were MS positive by ELISA, 15 were positive by SPA, 17 were positive by HI, and 18 were positive by both culture and PCR (3.19±1.74 MCN log 10) (see Table 13 for a summary of serology results).

Serology. The chickens tested for MS and MG pre-vaccination (5 WOA) were negative by for antibodies using SPA, HI, and ELISA. At 10 WOA (4 WPV) the samples from the non-vaccinated (NV) groups were all negative while the samples from the vaccinated groups were all MS positive by SPA and ELISA but only one (6.7%) of the samples from the vaccinated groups was positive by HI at that time with a titer of 1.30. At 21 WOA (15 WPV) chickens from the vaccinated groups were MS positive by ELISA (20/20), SPA (15/20), HI (17/20). The serum samples from the non-vaccinated groups were all negative.

At 4 WPC, all chickens in the negative control group (NV/NC) were negative for MS antibodies. There was no significant difference in the number of chickens that tested positive for MS and in titers of hens by SPA, HI, and ELISA among the other groups although the ELISA titer of the vaccinated-only group (V/NC) was the lowest.

At 6 WPC (29 WOA), the serology results were similar to those observed at 4 WPC as the negative controls (NV/NC) remained negative with all serological test and there were no significant differences in the number of MS positives and titers among the other groups. Fewer chickens were MS positive by SPA in the vaccinated-WVU 1853-challenged group (V/C-W) and K6677 challenged group (NV/C-K) (2/6 and 3/6 respectively), while the WVU 1853-challenged (NV/C-W and V/C-W) groups had the lowest SPA and ELISA titers. The serology results are summarized in see Table 13.

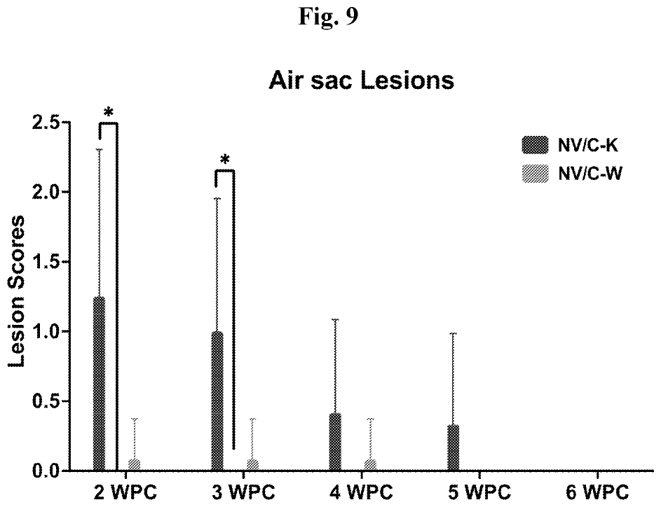

Air sac lesions. The severity of air sac lesions was highest at 2 WPC in the non-vaccinated group challenged with K6677 (NV/C-K) group, but the severity of lesion scores in this group declined progressively to zero at 6 WPC. Airsacculitis was significantly higher in NV/C-K group when compared to the non-vaccinated group challenged with WVU 1853 (NV/C-W) at 2 WPC but not at 3, 4, 5 or 6 WPC. Also, the severity of the airsacculitis was significantly higher in the non-vaccinated groups challenged with K6677 and WVU 1853 (NV/C-K and NV/C-W) when compared to the vaccinated and challenged groups (V/C-K and V/C-W) at 2 and 3 WPC. None of the three groups vaccinated with K5885 (V/C-K, V/C-W and V/NC) developed airsacculitis (FIG. 9).

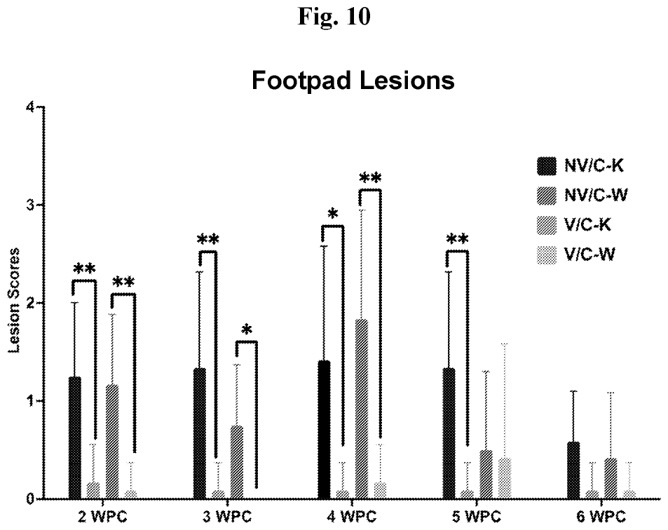

Footpad lesions. The number of chickens with foot pad lesions as well as the severity of footpad lesions were significantly higher in the non-vaccinated groups challenged with K6677 and WVU 1853 (NV/C-K and NV/C-W) in this study as compared to the three K5885 vaccinated groups (V/NC, V/C-K and V/C-W) at 2, 3, and 4 WPC. (P<0.05). There were no significant differences in the number of chickens with footpad lesions or the severity of lesions caused by K6677 when compared to the WVU 1853 in the non-vaccinated groups (NV/C-K and NV/C-W) (P<0.05) (FIG. 10).

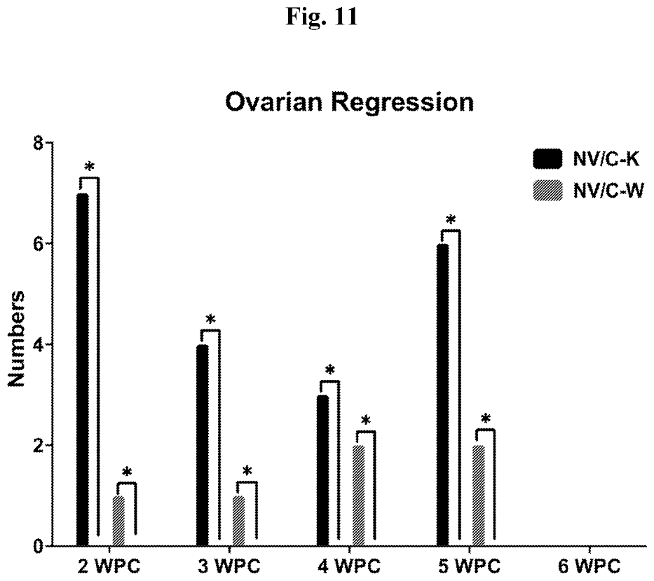

Ovarian Regression. Ovarian regression was not observed in the negative control group or any of the vaccinated groups including the groups challenged with K6677 or WVU 1853 (V/NC, V/C-K, V/C-W). The percentage of ovarian regression was higher in the K6677-challenged chickens than in the WVU 1853-challenged chickens, but the differences were not statistically significant (P<0.05) (FIG. 11).

MS Isolation. As shown in Tables 16-20, MS was isolated from the choanal cleft of at minimum 83% (10/12) of the chickens in both the WVU 1853 challenged groups (NV/C-W, V/C-W) at all time points post challenge; but in the K6677 challenged only group (NV/C-K), isolation rates of MS from the choanal cleft reduced from 100% (12/12) at 3 WPC to 33.3% (4/12) at 4 WPC and the 0% at 5 and 6 WPC. In the oviduct, MS was isolated in only one sample each from the 2 groups challenged with WVU 1853 (V/C-W, NV/C-W) at 2 WPC; and also, one sample from the vaccinated-only group (V/NC) at 4 WPC.

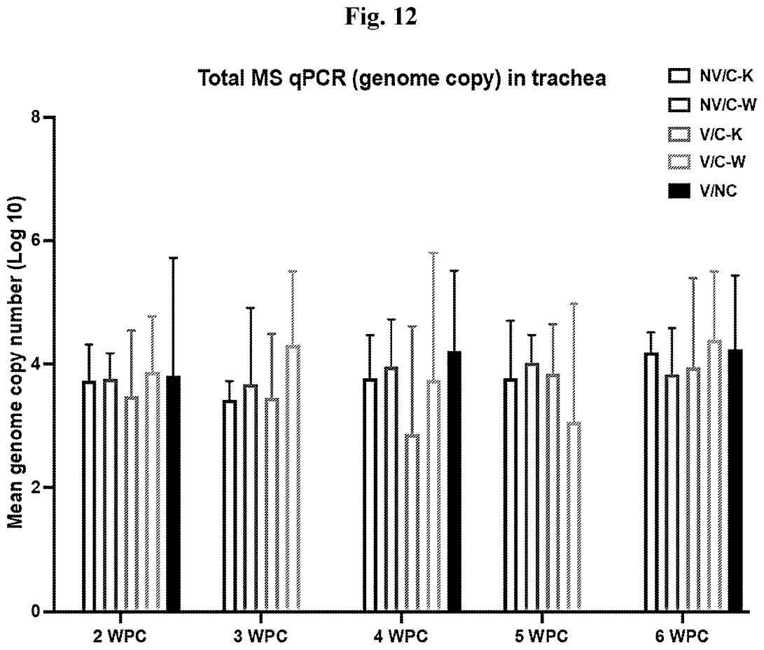

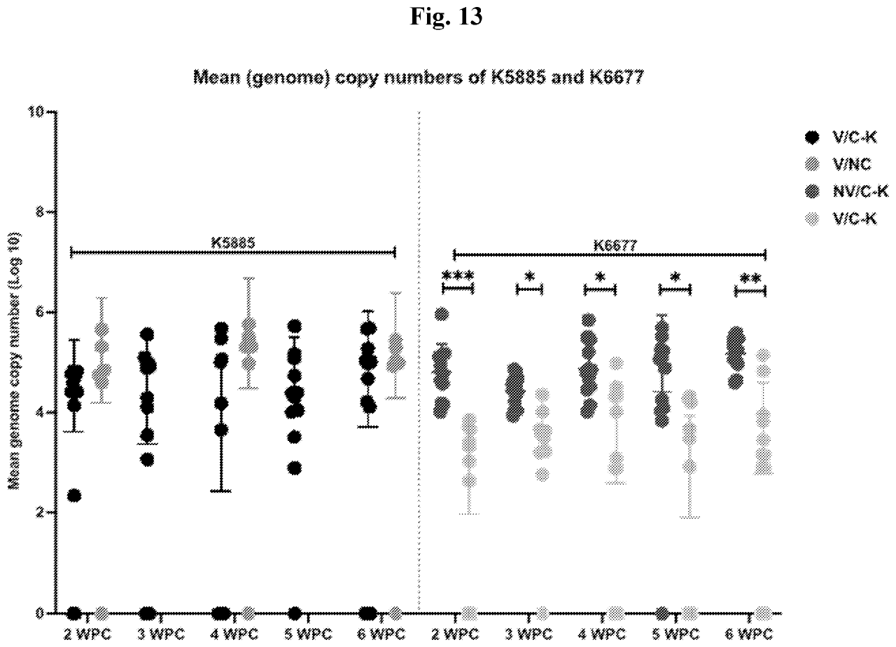

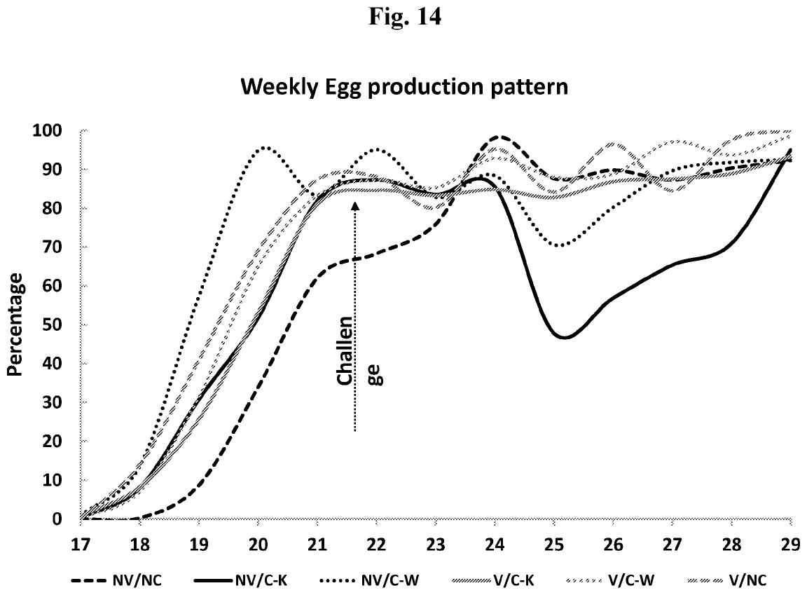

Real-time PCR (qPCR). At least 92% (11/12) of the trachea washes from chicken in all the groups, except the negative controls (NV/NC), were MS positive and there were no significant differences in the MS mean genome copy numbers (MCN log 10) among the positive groups (FIG. 12) (P<0.05). None of the oviduct samples from any of the groups was MS positive (Tables 16-20). As shown in Table 19, using K6677-specific PCR primers, the number of K6677-positive chickens was higher at 2, 3, 4, 5, and 6 WPC in the non-vaccinated K6677-challenged group (NV/C-K) when compared to the vaccinated K6677-challenged (V/C-K) but the differences were not statistically significant (P<0.05). However, there was a significantly higher K6677 mean genome copy number log 10 in the non-vaccinated group that was challenged with K6677 (NV/C-K) when compared to the vaccinated group that was challenged with K6677 (V/C-K) at 2, 3, 4, 5, and 6 WPC (P<0.05) (FIG. 13). On the other hand, in Table 20, using K5885-specific primers, there were higher K5885 mean genome copy numbers log 10 in the vaccinated group (V/NC) compared to the vaccinated group challenged with K6677 (V/C-K) but the difference was not significant (P<0.05). WVU 1853-specific PCR results are pending. Egg production. At 25 WOA (2 WPC), the percentage of weekly egg production was significantly reduced in the K6677 challenged group (NV/C-K) when compared to the negative control, vaccinated, and vaccinated groups (NV/NC, V/C-K, V/C-W) (P<0.05). There was also a significant reduction in the percentage of weekly egg production in the WVU 1853-challenged group (NV/C-W) when compared to the negative control, vaccinated then WVU 1853-challenged, and the vaccinated groups (NV/NC, V/C-W, V/NC) (Table 21). This significant reduction in the weekly egg production percentage continued for three weeks (3, 4, and 5 WPC) in the K6677 group (NV/C-K) until they were 28 WOA unlike in the WVU 1853-challenged group (NV/C-W), where the significant reduction was only at 3 WPC (P<0.05). Egg production in the K6677 challenged group (NV/C-K) was significantly lower than those of the WVU 1853 group (NV/C-W) at 26-, 27- and 28 WOA but at 29 WOA, egg production in all the groups was similar (P<0.05) (FIG. 14).

Egg Shell Strength test and Egg abnormalities. There was no significant difference (p<0.05) in the eggshell strength before and after the challenge with K6677 and WVU 1853. There was also no significant difference (p<0.05) in the eggshell strength in the non-vaccinated (NV/C-K and NV/C-W) groups when compared with the vaccinated (V/C-K and V/C-W) groups. Egg abnormalities did not differ significantly among the different groups after K6677 and WVU 1853 challenge when compared to the NV/NC and V/NC groups.

### DISCUSSION

The administration of a single dose of K5885 intra-ocularly stimulated immune responses in chickens at 4 weeks post-vaccination (WPV) as detected by circulating anti-MS antibodies by serology. The antibody response to K5885 vaccination increased from 4 WPV to higher levels at 15 WPV and titers were maintained to the end of the study at 24 WPV.

The colonization and replication rates of the challenge strains K6677 and WVU 1853 were similar in the tracheal washes as detected by qPCR from 2 WPC to 6 WPC, but the isolation rate from the choanal cleft was not similar between the strains. K6677 could not be isolated from challenged birds after 4 WPC, unlike WVU 1853 which was isolated up to 6 WPC. The K6677 strain was also not isolated or detected via qPCR from oviducts and despite the isolation of WVU 1853 and K5885 from a few oviducts, they could also not be detected via qPCR from the oviduct swabs.

Challenge with the K6677 strain resulted in more severe MS-associated lesions in chickens than the WVU 1853 strain. Airsacculitis, footpad lesions, and ovarian regression were all observed at higher levels in the K6677-challenged chickens compared to the WVU 1853-challenged group. Egg production was also significantly reduced in the K6677-challenged chickens compared to the WVU 1853-challenged chickens (P<0.05). This suggests that K6677 strain is more virulent than WVU 1853.

Vaccination with K5885 prevented MS-associated lesions in all of the vaccinated groups that were challenged with either K6677 or WVU 1853. K5885 vaccination also reduced the replication of K6677 in the trachea; this may be an explanation of the absence of MS associated lesions in the vaccinated groups.

Eggshell strength and egg abnormalities were not significantly affected in the groups that were challenged with K6677 and WVU 1853. As eggshell defects appears to be associated with specific strains of MS, the results of this study imply that neither of the MS strains used have a tropism for the reproductive tract and therefore do not cause eggshell apex abnormalities (Feberwee et al., 2009, Avian Pathology; 38(2):187-187; Ferguson-Noel et al., 2013, Mycoplasma synoviae infection. In D. E. Swayne, J. R. Glisson, L. R. McDougald, L. K. Nolan, D. L. Suarez, & V. Nair (Eds.), Diseases of Poultry (pp. 900-906). Wiley-Blackwell; and Landman, 2014, Avian Pathology; 43(1):2-8). In addition to the lack of effect on the eggshells, MS was not isolated from eggs from K6677 and WVU 1853-challenged chickens.

This study demonstrates that the vaccine candidate K5885 prevented MS associated lesions (including airsacculitis, ovarian regression and footpad lesions (synovitis)), reduced the shedding of MS and reduced egg production losses associated with virulent MS strain infection. Further studies should be carried out to evaluate the ability of K5885 to prevent eggshell apex abnormalities (EAA) caused by EAA-associated MS strains and understand the mechanism through which K5885 may confer protection from disease resulting from MS infection in chickens.

TABLE 11
Groups, Titers, and Doses of vaccine and challenge isolates per chicken.

| | | | Challenge | | Challenge | | |
|---|---|---|---|---|---|---|---|
| | Vaccinated | | Isolates Titer | Challenge | Isolates Titer | Challenge | Total Dose |
| | with | Challenge | (ccu per ml) | Method | (ccu per ml) | Method | per chicken |
| Groups | K5885 | Isolates | Day 1 | Day 1 | Day 2 | Day 2 | (log10) |
| NV/NC | No | No | No | No | No | No | No |
| NV/C-K | No | K6677 | 1.51 × 10⁷ | 1 ml aerosol | 2.34 × 10⁷ | 1 ml aerosol ± | 13.6 |
| | | | | | | 0.1 ml intra footpad | |
| NV/C-W | No | WVU 1853 | 6.6 × 10⁸ | 1 ml aerosol | 3.8 × 10⁸ | 1 ml aerosol ± | 17.4 |
| | | 1853 | | | | 0.1 ml intra footpad | |
| V/C-K | Yes | K6677 | 1.51 × 10⁷ | 1 ml aerosol | 2.34 × 10⁷ | 1 ml aerosol ± | 13.6 |
| | | | | | | 0.1 ml intra footpad | |
| V/C-W | Yes | WVU 1853 | 6.6 × 10⁸ | 1 ml aerosol | 3.8 × 10⁸ | 1 ml aerosol ± | 17.4 |
| | | 1853 | | | | 0.1 ml intra footpad | |
| V/NC | Yes | No | No | No | No | No | No |

NV/NC: non-vaccinated non- challenged
NV/C-K: non-vaccinated K6677-challenged
NV/C-W: non-vaccinated WVU 1853-challenged
V/C-K: vaccinated K6677-challenged
V/C-W: vaccinated WVU 1853-challenged
V/NC: vaccinated non-challenged

TABLE 12
Experimental Design and Procedure.

| | Age | | | | | |
|---|---|---|---|---|---|---|
| Date | (wk) | WPV | WPC | PDRC # | K # | Procedure |
| 12-Apr | 4 | −2 | −19 | | | Receipt of 318 chickens |
| 19-Apr | 5 | −1 | −18 | 144647 | K7109 | Pre-vaccination bleed and swab |
| 26-Apr | 6 | 0 | −17 | | K7110 | Intra-ocular vaccination (V/C-K, |
| | | | | | | V/C-W and V/NC groups) |
| 25-May | 10 | 4 | −13 | 145085 | K7115 | Post-vaccination bleed and swab |
| 9-Aug | 21 | 15 | −2 | 145809 | K7128 | Pre-challenge bleed and swab |
| 16-Aug | 22 | 16 | −1 | | | Eggshell strength evaluation for |
| | | | | | | 1 week (Alternate days) |
| 23-Aug | 23 | 17 | 0 | | | Aerosol challenge of K6677 and |
| | | | | | | WVU 1853 (NV/C-K, V/C-K, NV/C-W |
| | | | | | | and V/C-W groups) |
| 24-Aug | 23 | 18 | 0 | | | Aerosol and intra-footpad challenge |
| | | | | | | of K6677 and WVU 1853 (NV/C-K, |
| | | | | | | V/C-K, NV/C-W and V/C-W groups) |
| 26-Aug | 23 | 18 | 0 | | K7130-32; | Egg collection on alternate days |
| | | | | | K7134-37; | for incubation before culture |
| | | | | | K7139-41; | begins |
| | | | | | K7143-46; | |
| | | | | | K7148-50; | |
| | | | | | K7152-54 | |
| 6-Sep | 25 | 20 | 2 | 146207- | K7133A-F | Necropsy 1- 66 hens euthanized (12 |
| | | | | 146212 | | from all groups except V/NC- 6 hens) |
| 13-Sep | 26 | 21 | 3 | 146235- | K7138A-E | Necropsy 2- 60 hens euthanized (12 |
| | | | | 146239 | | from all groups) |
| 14-Sep | 26 | 21 | 3 | 146312- | K7142A-F | Eggshell strength evaluation for 1 |
| | | | | 146316 | | week on alternate days |
| 20-Sep | 27 | 22 | 4 | 146466- | K7147A-F | Necropsy 3- 66 hens euthanized (12 |
| | | | | 146470 | | from all groups except V/NC- 6 hens); |
| | | | | | | 6 hens bled and swabbed |
| 27-Sep | 28 | 23 | 5 | 146466- | K7147A-E | Necropsy 4- 60 hens euthanized (12 |
| | | | | 146470 | | from all groups) |
| 4-Oct | 29 | 24 | 6 | 146526- | K7151A-F | Necropsy 5- 66 hens euthanized (12 |
| | | | | 146531 | | from all groups except V/NC- 6 hens); |
| | | | | | | 6 hens bled and swabbed |

TABLE 13
Serology results from vaccinated, non-vaccinated, challenged,
and non-challenged groups at 5, 10, 21, 27 and 29 WOA.^{A}

| Time | Vaccination | Challenge | | | |
|---|---|---|---|---|---|
| points | Status | Status | SPA | HI | ELISA |
| Pre- | NV | NC | 0/30^{B} | 0/30 | 0/30 |
| vaccination | | | (0.0)^{C} | (0.0)^{D} | (0.0)^{E} |
| (5 WOA) | NV | NC | 0/30 | 0/30 | 0/30 |
| | | | (0.0) | (0.0) | (0.0) |
| Post- | NV | NC | 0/15 | 0/15 | 0/15 |
| vaccination | | | (0.0) | (0.0) | (0.0) |
| (10 WOA) | V | NC | 15/15 | 1/15 | 14/15 |
| | | | (1.4 ± 0.5) | (0.1 ± 0.3) | (0.3 ± 0.4) |
| Pre- | NV | NC | 0/10 | 0/10 | 0/10 |
| challenge | | | (0.0) | (0.0) | (0.0) |
| (21 WOA) | V | NC | 15/20 | 17/20 | 20/20 |
| | | | (0.8 ± 0.4) | (1.2 ± 0.6) | (1.2 ± 0.8) |
| Post- | NV | NC | 0/12^{B} | 0/12 | 0/12 |
| challenge | | | (0.0 ± 0.0) | (0.0) | (0.0) |
| (4 WPC; | NV | C | 5/6 | 6/6 | 6/6 |
| 27 WOA) | | (K6677) | (0.8 ± 0.4) | (1.7 ± 0.2) | (3.9 ± 1.5) |
| | NV | C | 6/6 | 6/6 | 6/6 |
| | | (WVU 1853) | (1.0 ± 0.0) | (1.8 ± 0.2) | (5.3 ± 0.7) |
| | V | C | 5/6 | 6/6 | 6/6 |
| | | (K6677) | (0.8 ± 0.4) | (1.7 ± 0.1) | (4.4 ± 1.1) |
| | V | C | 6/6 | 6/6 | 6/6 |
| | | (WVU 1853) | (1.2 ± 0.4) | (1.6 ± 0.0) | (5.3 ± 1.3) |
| | V | NC | 5/6 | 5/6 | 6/6 |
| | | | (1.3 ± 0.8) | (1.6 ± 0.0) | (2.7 ± 0.9) |
| Post- | NV | NC | 0/12 | 0/12 | 0/12 |
| challenge | | | (0.0 ± 0.0) | (0.0 ± 0.0) | (0.0 ± 0.0) |
| (6 WPC; | NV | C | 5/6 | 6/6 | 6/6 |
| 29 WOA) | | (K6677) | (1.0 ± 0.6) | (2.0 ± 0.1) | (1.5 ± 0.8) |
| | NV | C | 2/6 | 6/6 | 6/6 |
| | | (WVU 1853) | (0.3 ± 0.5) | (1.8 ± 0.2) | (1.3 ± 0.2) |
| | V | C | 3/6 | 6/6 | 6/6 |
| | | (K6677) | (0.5 ± 0.5) | (1.9 ± 0.1) | (2.8 ± 0.8) |
| | V | C | 5/6 | 6/6 | 6/6 |
| | | (WVU 1853) | (1.2 ± 0.8) | (1.7 ± 0.2) | (2.0 ± 1.1) |
| | V | NC | 6/6 | 5/6 | 6/6 |
| | | | (1.5 ± 0.5) | (1.8 ± 0.2) | (1.6 ± 0.7) |

NV—Non-vaccinated;
V—Vaccinated
NC—Non-challenged;
C—Challenged
^{A}Values within a column at a specific time point with a different lower-case superscript are significantly different (P ≤ 0.05);
^{B}No. of positive samples/No. of tested samples (SPA: ≥1, HI: ≥40, and ELISA: ≥0.5);
^{C}Mean agglutination grade ± SD (from 0 to 4);
^{D}Mean titer log10 ± SD
^{E}Mean sample/positive ratio ± SD.

TABLE 14
Lesion scores, MS isolation, and qPCR from chickens at 2 weeks post challenge^{A}

| | Air sac | Ov | Footpad | CC | Tr MS | Oviduct | Oviduct |
|---|---|---|---|---|---|---|---|
| Group | Lesions | Rgr | Lesion | Culture | MCNlog10 | Culture | MS MCNlog10 |
| NV/NC | 0/12^{B} | 0/12 | 0/12 | 0/12 | 0/12 | 0/12 | 0/12 |
| | (0.0 ± 0.0)^{bC} | | (0.0 ± 0.0)^{bC} | | (0.0 ± 0.0)^{aD} | | (0.0 ± 0.0)^{D} |
| NV/C-K | 8/12 | 7/12 | 10/12 | 11/12 | 12/12 | 0/12 | 0/12 |
| | (1.3 ± 1.1)^{a} | | (1.3 ± 0.8)^{a} | | (3.7 ± 0.59)^{b} | | (0.0 ± 0.0) |
| NV/C-W | 1/12 | 1/12 | 10/12 | 12/12 | 12/12 | 1/12 | 0/12 |
| | (0.1 ± 0.3)^{b} | | (1.2 ± 0.7)^{a} | | (3.7 ± 0.4)^{b} | | (0.0 ± 0.0) |
| V/C-K | 0/12 | 0/12 | 2/12 | 10/12 | 12/12 | 0/12 | 0/12 |
| | (0.0 ± 0.0)^{b} | | (0.2 ± 0.4)^{b} | | (3.5 ± 1.1)^{b} | | (0.0 ± 0.0) |
| V/C-W | 0/12 | 0/12 | 1/12 | 12/12 | 12/12 | 1/12 | 0/12 |
| | (0.0 ± 0.0)^{b} | | (0.1 ± 0.3)^{b} | | (3.9 ± 0.9)^{b} | | (0.0 ± 0.0) |
| V/NC | 0/6 | 0/6 | 1/6 | 5/6 | 5/6 | 0/6 | 0/6 |
| | (0.0 ± 0.0) | | (0.2 ± 0.4) | | (3.8 ± 1.9) | | (0.0 ± 0.0) |

Ov Rgr—Ovarian Regression
CC—Choanal cleft
Tr—Trachea
NV/NC: non-vaccinated non- challenged
NV/C-K: non-vaccinated K6677-challenged
NV/C-W: non-vaccinated WVU 1853-challenged
V/C-K: vaccinated K6677-challenged
V/C-W: vaccinated WVU 1853-challenged
V/NC: vaccinated non-challenged
^{A}Values within a column with a different lower-case superscript are significantly different (P ≤ 0.05);
^{B}No. of positive samples/No. of tested samples (Air sac score ≥1; Footpad score ≥1; Ovarian regression ≥1);
^{C}Macroscopically scored from 0 to 4;
^{D}Mean (genome) copy number Log 10 ± SD.

TABLE 15
Lesion scores, MS isolation, and qPCR from chickens at 3 weeks post challenge^{A}

| | Air sac | Ov | Footpad | CC | Tr MS | Oviduct | Oviduct |
|---|---|---|---|---|---|---|---|
| Group | Lesions | Rgr | Lesion | Culture | MCNlog10 | Culture | MS MCNlog10 |
| NV/NC | 0/12^{B} | 0/12 | 0/12 | 0/12 | 0/12 | 0/12 | 0/12 |
| | (0.0 ± 0.0) ^{bC} | | (0.0 ± 0.0) ^{bC} | | (0.0 ± 0.0) ^{bD} | | (0.0 ± 0.0) ^{D} |
| NV/C-K | 8/12 | 4/12 | 10/12 | 12/12 | 12/12 | 0/12 | 0/12 |
| | (1.0 ± 1.0) ^{a} | | (1.0 ± 1.3) ^{a} | | (3.4 ± 0.3) ^{a} | | (0.0 ± 0.0) |
| NV/C-W | 1/12 | 1/12 | 8/12 | 10/12 | 11/12 | 0/12 | 0/12 |
| | (0.1 ± 0.3) ^{b} | | (0.8 ± 0.6) ^{a} | | (3.7 ± 1.2) ^{a} | | (0.0 ± 0.0) |
| V/C-K | 0/12 | 0/12 | 1/12 | 11/12 | 12/12 | 0/12 | 0/12 |
| | (0.0 ± 0.0) ^{b} | | (0.1 ± 0.3) ^{b} | | (3.4 ± 1.0) ^{a} | | (0.0 ± 0.0) |
| V/C-W | 0/12 | 0/12 | 0/12 | 6/12 | 12/12 | 0/12 | 0/12 |
| | (0.0 ± 0.0) ^{b} | | (0.0 ± 0.0) ^{b} | | (4.3 ± 1.2) ^{a} | | (0.0 ± 0.0) |

Ov Rgr—Ovarian Regression
CC—Choanal cleft
Tr—Trachea
NV/NC: non-vaccinated non- challenged
NV/C-K: non-vaccinated K6677-challenged
NV/C-W: non-vaccinated WVU 1853-challenged
V/C-K: vaccinated K6677-challenged
V/C-W: vaccinated WVU 1853-challenged
V/NC: vaccinated non-challenged
^{A}Values within a column with a different lower-case superscript are significantly different (P ≤ 0.05);
^{B}No. of positive samples/No. of tested samples (Air sac score ≥1; Footpad score ≥1; Ovarian regression ≥1);
^{C} Macroscopically scored from 0 to 4;
^{D} Mean (genome) copy number Log 10 ± SD.

TABLE 16
Lesion scores, MS isolation, and qPCR from chickens at 4 weeks post challenge^{A}

| | Air sac | Ov | Footpad | CC | Tr MS | Oviduct | Oviduct |
|---|---|---|---|---|---|---|---|
| Group | Lesions | Rgr | Lesion | Culture | MCNlog10 | Culture | MS MCNlog10 |
| NV/NC | 0/12^{B} | 0/12 | 0/12 | 0/12 | 0/12 | 0/12 | 0/12 |
| | (0.0 ± 0.0)^{aC} | | (0.0 ± 0.0)^{bC} | | (0.0 ± 0.0)^{bD} | | (0.0 ± 0.0)^{D} |
| NV/C-K | 4/12 | 3/12 | 9/12 | 4/12 | 12/12 | 0/12 | 0/12 |
| | (0.4 ± 0.7)^{a} | | (1.4 ± 1.2)^{a} | | (3.78 ± 0.70)^{a} | | (0.0 ± 0.0) |
| NV/C-W | 1/12 | 2/12 | 12/12 | 12/12 | 12/12 | 0/12 | 0/12 |
| | (0.1 ± 0.3)^{a} | | (1.8 ± 1.1)^{a} | | (4.0 ± 0.78)^{a} | | (0.0 ± 0.0) |
| V/C-K | 0/12 | 0/12 | 1/12 | 9/12 | 12/12 | 0/12 | 0/12 |
| | (0.0 ± 0.0)^{a} | | (0.1 ± 0.3)^{b} | | (2.2 ± 1.7)^{a} | | (0.0 ± 0.0) |
| V/C-W | 0/12 | 0/12 | 2/12 | 10/12 | 11/12 | 0/12 | 0/12 |
| | (0.0 ± 0.0)^{a} | | (0.2 ± 0.4)^{b} | | (3.7 ± 2.1)^{a} | | (0.0 ± 0.0) |
| V/NC | 0/12 | 0/6 | 0/6 | 6/6 | 6/6 | 1/6 | 0/6 |
| | (0.0 ± 0.0) | | (0.0 ± 0.0) | | (4.2 ± 1.3) | | (0.0 ± 0.0) |

Ov Rgr—Ovarian Regression
CC—Choanal cleft
Tr—Trachea
NV/NC: non-vaccinated non- challenged
NV/C-K: non-vaccinated K6677-challenged
NV/C-W: non-vaccinated WVU 1853-challenged
V/C-K: vaccinated K6677-challenged
V/C-W: vaccinated WVU 1853-challenged
V/NC: vaccinated non-challenged
^{A}Values within a column with a different lower-case superscript are significantly different (P ≤ 0.05);
^{B}No. of positive samples/No. of tested samples (Air sac score ≥1; Footpad score ≥1; Ovarian regression ≥1);
^{C}Macroscopically scored from 0 to 4;
^{D}Mean (genome) copy number Log 10 ± SD.

TABLE 17
Lesion scores, MS isolation, and qPCR from chickens at 5 weeks post challenge^{A}

| | Air sac | Ov | Footpad | CC | Tr MS | Oviduct | Oviduct |
|---|---|---|---|---|---|---|---|
| Group | Lesions | Rgr | Lesion | Culture | MCNlog10 | Culture | MS MCNlog10 |
| NV/NC | 0/12^{B} | 0/12 | 0/12 | 0/12 | 0/12 | 0/12 | 0/12 |
| | (0.0 ± 0.0)^{aC} | | (0.0 ± 0.0)^{bC} | | (0.0 ± 0.0)^{bD} | | (0.0 ± 0.0)^{D} |
| NV/C-K | 3/12 | 6/12 | 10/12 | 0/12 | 12/12 | 0/12 | 2/12 |
| | (0.3 ± 0.7)^{a} | | (1.3 ± 1.0)^{a} | | (3.7 ± 0.9)^{a} | | (0.1 ± 0.2) |
| NV/C-W | 0/12 | 2/12 | 4/12 | 12/12 | 12/12 | 0/12 | 0/12 |
| | (0.0 ± 0.0)^{a} | | (0.5 ± 0.8)^{a} | | (4.0 ± 0.5)^{a} | | (0.0 ± 0.0) |
| V/C-K | 0/12 | 0/12 | 1/12 | 11/12 | 12/12 | 0/12 | 0/12 |
| | (0.0 ± 0.0)^{a} | | (0.1 ± 0.3)^{b} | | (3.8 ± 0.8)^{a} | | (0.0 ± 0.0) |
| V/C-W | 0/12 | 0/12 | 2/12 | 11/12 | 10/12 | 0/12 | 0/12 |
| | (0.0 ± 0.0)^{a} | | (1.2 ± 0.4)^{a} | | (3.1 ± 1.9)^{a} | | (0.0 ± 0.0) |

Ov Rgr—Ovarian Regression
CC—Choanal cleft
Tr—Trachea
NV/NC: non-vaccinated non- challenged
NV/C-K: non-vaccinated K6677-challenged
NV/C-W: non-vaccinated WVU 1853-challenged
V/C-K: vaccinated K6677-challenged
V/C-W: vaccinated WVU 1853-challenged
V/NC: vaccinated non-challenged
^{A}Values within a column with a different lower-case superscript are significantly different (P ≤ 0.05);
^{B}No. of positive samples/No. of tested samples (Air sac score ≥1; Footpad score ≥1; Ovarian regression ≥1);
^{C}Macroscopically scored from 0 to 4;
^{D}Mean (genome) copy number Log 10 ± SD.

TABLE 18
Lesion scores, MS isolation, and qPCR from chickens at 6 weeks post challenge^{A}

| | Air sac | Ov | Footpad | CC | Tr MS | Oviduct | Oviduct |
|---|---|---|---|---|---|---|---|
| Group | Lesions | Rgr | Lesion | Culture | MCNlog10 | Culture | MS MCNlog10 |
| NV/NC | 0/12^{B} | 0/12 | 0/12 | 0/12 | 0/12 | 0/12 | 0/12 |
| | (0.0 ± 0.0)^{C} | | (0.0 ± 0.0)^{bC} | | (0.0 ± 0.0) ^{bD} | | (0.0 ± 0.0)^{D} |
| NV/C-K | 0/12 | 0/12 | 7/12 | 0/12 | 12/12 | 0/12 | 0/12 |
| | (0.0 ± 0.0) | | (0.6 ± 0.5)^{a} | | (4.1 ± 0.3)^{a} | | (0.0 ± 0.0) |
| NV/C-W | 0/12 | 0/12 | 4/12 | 12/12 | 12/12 | 0/12 | 0/12 |
| | (0.0 ± 0.0) | | (0.4 ± 0.7)^{a} | | (3.8 ± 0.8)^{a} | | (0.0 ± 0.0) |
| V/C-K | 0/12 | 0/12 | 1/12 | 10/12 | 12/12 | 0/12 | 0/12 |
| | (0.0 ± 0.0) | | (0.1 ± 0.3)^{a} | | (3.9 ± 1.5)^{a} | | (0.0 ± 0.0) |
| V/C-W | 0/12 | 0/12 | 1/12 | 12/12 | 12/12 | 0/12 | 0/12 |
| | (0.0 ± 0.0) | | (0.1 ± 0.3)^{a} | | (4.4 ± 1.2)^{a} | | (0.0 ± 0.0) |
| V/NC | 0/6 | 0/6 | 0/6 | 5/6 | 6/6 | 0/6 | 0/6 |
| | (0.0 ± 0.0) | | (0.0 ± 0.0) | | (4.2 ± 1.2) | | (0.0 ± 0.0) |

Ov Rgr—Ovarian Regression
CC—Choanal cleft
Tr—Trachea
NV/NC: non-vaccinated non- challenged
NV/C-K: non-vaccinated K6677-challenged
NV/C-W: non-vaccinated WVU 1853-challenged
V/C-K: vaccinated K6677-challenged
V/C-W: vaccinated WVU 1853-challenged
V/NC: vaccinated non-challenged
^{A}Values within a column with a different lower-case superscript are significantly different (P ≤ 0.05);
^{B}No. of positive samples/No. of tested samples (Air sac score ≥1; Footpad score ≥1; Ovarian regression ≥1);
^{C}Macroscopically scored from 0 to 4;
^{D}Mean (genome) copy number Log 10 ± SD.

TABLE 19
qPCR of K6677 in the NV/C-K and V/C-K groups
from 2 to 6 weeks post challenge^{A}

| Groups | | 2 WPC | 3 WPC | 4 WPC | 5 WPC | 6 WPC |
|---|---|---|---|---|---|---|
| K6677 | NV/C-K | 12/12^{B} | 12/12 | 12/12 | 12/12 | 12/12 |
| qPCR | | (4.8 ± 0.5)^{aC} | (4.4 ± 0.3) | (4.9 ± 0.6) | (4.4± 1.5) | (5.2± 0.3) |
| | V/C-K | 7/12 | 11/12 | 8/12 | 6/12 | 9/12 |
| | | (2.4 ± 1.9) | (2.8 ±1.4) | (1.8±2.1) | (1.3 ± 2.0) | (3.1 ± 1.7) |

NV/NC: non-vaccinated non- challenged
NV/C-K: non-vaccinated K6677-challenged
NV/C-W: non-vaccinated WVU 1853-challenged
V/C-K: vaccinated K6677-challenged
V/C-W: vaccinated WVU 1853-challenged
V/NC: vaccinated non-challenged
^{A}Values within a column with a different lower-case superscript are significantly different (P ≤ 0.05);
^{B}No. of positive samples/No. of tested samples (Air sac score ≥1; Footpad score ≥1; Ovarian regression ≥1);
^{C}Mean (genome) copy number Log 10 ± SD.

TABLE 20
qPCR of K5885 in the V/C-K and V/NC groups from 2 to 6 weeks post challenge.

| Groups | | 2 WPC | 3 WPC | 4 WPC | 5 WPC | 6 WPC |
|---|---|---|---|---|---|---|
| K5885 | V/C-K | 10/12^{A} | 9/12 | 9/12 | 11/12 | 9/12 |
| qPCR | | (3.6 ± 1.8)^{B} | (3.4 ± 2.1) | (2.4 ± 2.6) | (4.0 ± 1.5) | (3.7 ± 2.3) |
| | V/NC | 5/6 | | 5/6 | | 5/6 |
| | | (4.2 ± 2.1) | | (4.5 ± 2.2) | | (4.3 ± 2.1) |

NV/NC: non-vaccinated non- challenged
NV/C-W: non-vaccinated WVU 1853-challenged
V/C-W: vaccinated WVU 1853-challenged
NV/C-K: non-vaccinated K6677-challenged
V/C-K: vaccinated K6677-challenged
V/NC: vaccinated non-challenged
^{A}No. of positive samples/No. of tested samples (Air sac score ≥ 1; Footpad score ≥ 1; Ovarian regression ≥ 1);
^{B}Mean (genome) copy number Log 10 ± SD.

TABLE 21
Percentage of weekly egg production (with standard deviation values) of
the chickens in all groups from the onset of egg production to 6 WPC^{A}.

| Weeks | 17 | 18 | 19 | 20 | 21 | 22 | 23 | 24 | 25 | 26 | 27 | 28 | 29 |
|---|---|---|---|---|---|---|---|---|---|---|---|---|---|
| NV/NC | 0.0^{B} ± | 0.2 ± | 8.7 ± | 33.9 ± | 62.0 ± | 68.3 ± | 75.8 ± | 98.1 ± | 87.5 ± | 89.8 ± | 87.2 ± | 90.2 ± | 92.3 ± |
| | 0.0^{C} | 0.1 | 3.1 | 10.4 | 17.5^{b} | 16.6^{b} | 20.5^{b} | 23.8^{a} | 23.7^{a} | 28.5^{a} | 26.8^{a} | 32.5^{a} | 23.8^{a} |
| NV/C-K | 0.2 ± | 8.0 ± | 30.8 ± | 51.7 ± | 81.9 ± | 87.2 | 83.4 ± | 85.4 ± | 47.7 ± | 56.8 ± | 65.3 ± | 70.9 ± | 95.0 ± |
| | 0.1 | 2.6 | 9.9 | 14.7 | 17.5^{ab} | 22.6^{a} | 22.6^{a} | 22.3^{a} | 17.4^{d} | 15.4^{c} | 19.3^{c} | 20.8^{b} | 12.3^{a} |
| NV/C-W | 0.9 ± | 13.6 ± | 57.3 ± | 94.8 ± | 83.2 ± | 95.0 ± | 82.8 ± | 88.3 ± | 70.5 ± | 80.2 ± | 89.6 ± | 91.8 ± | 92.5 ± |
| | 0.4 | 4.2 | 18.6 | 27.8 | 17.5^{a} | 24.0^{a} | 24.1^{a} | 21.9^{a} | 19.8^{cd} | 25.4^{b} | 25.2^{a} | 29.4^{a} | 20.4^{a} |
| V/C-K | 0.9 ± | 8.2 ± | 25.6 ± | 53.1 ± | 81.0 ± | 84.6 ± | 83.2 ± | 84.8 ± | 82.7 ± | 86.8 ± | 87.5 ± | 88.8 ± | 93.3 ± |
| | 0.3 | 2.4 | 7.9 | 14.6 | 17.5^{ab} | 21.9^{a} | 23.1^{a} | 21.3^{a} | 22.2^{bc} | 28.2^{a} | 25.2^{a} | 27.3^{a} | 24.3^{a} |
| V/C-W | 0.2 ± | 7.1 ± | 31.4 ± | 64.9 ± | 83.2 ± | 87.2 ± | 85.2 ± | 92.8 ± | 87.9 ± | 88.8 ± | 97.0 ± | 93.6 ± | 98.6 ± |
| | 0.1 | 2.5 | 10.1 | 17.9 | 17.5^{a} | 22.3^{a} | 24.0^{a} | 22.8^{a} | 24.7^{a} | 28.1^{a} | 27.6^{a} | 32.0^{a} | 21.3^{a} |
| V/NC | 0.0 ± | 14.0 ± | 41.0 ± | 69.0 ± | 87.3 ± | 88.1 ± | 80.1 ± | 95.2 ± | 84.1 ± | 96.4 ± | 84.5 ± | 97.6 ± | 100.0 ± |
| | 0.0 | 4.9 | 11.9 | 18.7 | 17.5^{a} | 23.9^{a} | 23.4^{a} | 22.9^{a} | 25.3^{ab} | 33.8^{a} | 17.6^{b} | 45.1^{a} | 4.5^{a} |

NV/NC: non-vaccinated non- challenged
NV/C-K: non-vaccinated K6677-challenged
NV/C-W: non-vaccinated WVU 1853-challenged
V/C-K: vaccinated K6677-challenged
V/C-W: vaccinated WVU 1853-challenged
V/NC: vaccinated non-challenged
^{A}Values within a column with a different lower-case superscript are significantly different (P ≤ 0.05);
^{B}Percentage of weekly egg production;
^{C}Standard Deviation.

TABLE 22
Eggshell strength at 1-week pre-challenge and 3 weeks post challenge^{A}

| | 7 | 5 | 3 | 1 | 21 | 23 | 25 | 27 | 29 | 31 | 35 | 37 | 39 |
|---|---|---|---|---|---|---|---|---|---|---|---|---|---|
| | DPrC | DPrC | DPrC | DPrC | DPC | DPC | DPC | DPC | DPC | DPC | DPC | DPC | DPC |
| NV/ NC | 5.30^{A} ± | 5.28 ± | 5.28 ± | 5.50 ± | 5.25 ± | 5.40 ± | 5.41 ± | 4.96 ± | 5.19 ± | 5.16 ± | 5.09 ± | 5.31 ± | 5.29 ± |
| | 0.84^{B} | 1.10 | 0.83 | 0.84 | 1.63^{a} | 0.83^{a} | 0.87^{a} | 0.92^{a} | 0.66^{a} | 0.73^{a} | 1.29^{a} | 0.95^{a} | 0.63^{a} |
| NV/C-K | 5.41 ± | 5.28 ± | 5.65 ± | 5.51 ± | 5.25 ± | 4.99 ± | 5.33 ± | 5.70 ± | 5.28 ± | 5.03 ± | 5.59 ± | 4.26 ± | 5.75 ± |
| | 1.07 | 1.10 | 1.02 | 1.02 | 0.66^{a} | 0.85^{a} | 0.73^{a} | 0.64^{a} | 0.31^{a} | 1.40^{a} | 0.61^{a} | 0.74^{a} | 0.85^{a} |
| NV/C-W | 5.41 ± | 5.28 ± | 5.63 ± | 5.53 ± | 5.15 ± | 5.55 ± | 5.50 ±1 | 5.40 ± | 5.55 ± | 5.42 ± | 5.90 ± | 5.63 ± | 5.80 ± |
| | 1.07 | 1.10 | 0.72 | 0.84 | 0.84^{a} | 1.06^{a} | 0.93^{a} | 0.67^{a} | 0.74^{a} | 0.56^{a} | 0.56^{a} | 0.71^{a} | 0.67^{a} |
| V/C-K | 5.24 ± | 5.28 ± | 5.25 ± | 4.94 ± | 5.42 ± | 5.60 ± | 5.35 ± | 5.37 ± | 4.52 ± | 5.52 ± | 5.47 ± | 5.49 ± | 5.43 ± |
| | 0.91 | 0.92 | 1.05 | 0.91 | 0.96^{a} | 0.69^{a} | 0.92^{a} | 0.95^{a} | 1.26^{a} | 0.56^{a} | 0.58^{a} | 0.63^{a} | 0.66^{a} |
| V/C-W | 5.24 ± | 5.28 ± | 5.35 ± | 5.41 ± | 5.49 ± | 5.35 ± | 5.54 ± | 5.40 ± | 5.21 ± | 5.28 ± | 5.47 ± | 5.65 ± | 5.38 ± |
| | 0.91 | 0.92 | 0.91 | 0.86 | 0.76^{a} | 0.87^{a} | 0.68^{a} | 0.85^{a} | 0.84^{a} | 0.83^{a} | 0.88^{a} | 0.94^{a} | 0.60^{a} |
| V/NC | 5.24 ± | 5.28 ± | 4.1 ± | 5.64 ± | 5.38 ± | 5.62 ± | 5.64 ± | 5.34 ± | 4.29 ± | 4.95 ± | 4.86 ± | 5.51 ± | 5.03 ± |
| | 0.91 | 0.92 | 0.91 | 0.52 | 0.83^{a} | 0.71^{a} | 0.44^{a} | 0.89^{a} | 0.55^{a} | 0.55^{a} | 0.51^{a} | 0.70^{a} | 0.67^{a} |

DPrC: Days pre-challenge
DPC: Days post-challenge
NV/NC: non-vaccinated non- challenged
NV/C-K: non-vaccinated K6677-challenged
NV/C-W: non-vaccinated WVU 1853-challenged
V/C-K: vaccinated K6677-challenged
V/C-W: vaccinated WVU 1853-challenged
V/NC: vaccinated non-challenged
^{A}Average egg strength;
^{B}Standard Deviation

TABLE 23
Total egg abnormalities recorded^{A}

| Group | Broken | Misshapen | Total |
|---|---|---|---|
| NV/NC | 5 | 2 | 7 |
| NV/C-K | 2 | 4 | 6 |
| NV/C-W | 6 | 1 | 7 |
| V/C-K | 2 | 0 | 1 |
| V/C-W | 7 | 2 | 9 |
| V/NC | 1 | 0 | 0 |

NV/NC: non-vaccinated non-challenged
NV/C-W: non-vaccinated WVU 1853-challenged
V/C-W: vaccinated WVU 1853-challenged
NV/C-K: non-vaccinated K6677-challenged
V/C-K: vaccinated K6677-challenged
V/NC: vaccinated non-challenged

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. An isolated Mycoplasma synoviae strain, wherein the isolated Mycoplasma synoviae strain is the K5885 Mycoplasma synoviae strain deposited at the ATCC under Patent Designation PTA-127167.

2. (canceled)

3. (canceled)

4. A composition comprising the isolated Mycoplasma synoviae of claim 1.

5. The composition of claim 4 comprising water.

6. The composition of claim 4 comprising a pharmaceutically acceptable carrier.

7. The composition of claim 4 comprising an adjuvant.

8. (canceled)

9. The composition of claim 4, wherein the composition is lyophilized, freeze dried, frozen, or an effervescent tablet.

10. The composition of claim 4, wherein the composition is formulated for intranasal, intraocular, oral, mucosal, intramuscular, subcutaneous, or in ovo administration or for spraying or aerosolizing.

11. A vaccine comprising the isolated Mycoplasma synoviae of claim 1.

12. (canceled)

13. (canceled)

14. The vaccine of claim 11, wherein the vaccine is lyophilized, freeze dried, frozen, or an effervescent tablet.

15. (canceled)

16. A live vaccine for birds of the order Galliformes, the vaccine comprising an amount of the K5885 Mycoplasma synoviae strain deposited at the ATCC under Patent Deposit Designation PTA-127167 or a progeny or derivative thereof, sufficient to protect the birds from disease induced by Mycoplasma synoviae, and a pharmaceutically acceptable carrier.

17. A kit comprising the isolated Mycoplasma synoviae of claim 1 and printed instructions, wherein the contents of the kit are contained within packaging material.

18. (canceled)

19. (canceled)

20. A method of producing an immune response to Mycoplasma synoviae in a bird, the method comprising administering the isolated Mycoplasma synoviae of claim 1 to the bird.

21. A method for reducing susceptibility of a bird against disease induced by Mycoplasma synoviae, the method comprising administering the isolated Mycoplasma synoviae of claim 1 to the bird.

22. A method for protecting a bird against Mycoplasma synoviae infection, the method comprising administering the isolated Mycoplasma synoviae of claim 1 to the bird.

23. A method of reducing one or more clinical signs induced by a Mycoplasma synoviae infection in a bird, the method comprising administering an effective amount of the isolated Mycoplasma synoviae of claim 1 to the bird.

24. A method of producing an immune response to Mycoplasma synoviae in a bird, the method comprising administering the composition of claim 4 to the bird.

25-28. (canceled)

29. A method for reducing susceptibility of a bird against disease induced by Mycoplasma synoviae, the method comprising administering the vaccine of claim 11 to the bird.

30. (canceled)

31. (canceled)

32. The method of claim 20, wherein administration is intranasal, intraocular, oral, mucosal, intramuscular, subcutaneous, or in ovo or by eye drop, by aerosol, or by drinking water.

33. (canceled)

34. The method of claim 20, wherein the bird comprises a bird of the order Galliformes.

35. The method of claim 20, wherein the bird comprises a chicken or a turkey.
